(19) United States
(12) Patent Application Publication
(10) Pub. No.: US 2026/0337819 A1
(43) Pub. Date: Oct. 29, 2026

(54) METHODS AND SYSTEMS FOR GENE MUTAGENESIS

(71) Applicant: President and Fellows of Harvard College, Cambridge, MA (US)

(72) Inventors: David A. Weitz, Cambridge, MA (US); Karla Milcic, Cambridge, MA (US); Xinge Zhang, Cambridge, MA (US); Anqi Chen, Cambridge, MA (US)

(73) Assignee: President and Fellows of Harvard College, Cambridge, MA (US)

(21) Appl. No.: 19/115,024

(22) PCT Filed: Sep. 25, 2023
(86) PCT No.: PCT/US2023/075063

§ 371 Date: Mar. 25, 2025

Related U.S. Application Data
(60) Provisional application No. 63/410,116, filed on Sep. 26, 2022.
(60) Provisional application No. 63/410,140, filed on Sep. 26, 2022.

(51) Int. Cl. C40B 40/06; C12N 15/10

(52) U.S. Cl. CPC C40B 40/06; C12N 15/1031

(57) ABSTRACT

Systems and methods, and associated compositions, for producing large libraries of nucleic acids are generally described. The methods provided herein may be used to produce libraries that include mutations that are unlikely in conventional library generation techniques, and may facilitate the production of large libraries with reduced processing demands.

### RELATED APPLICATIONS

This application claims the benefit of U.S. Provisional Patent Application Ser. No. 63/410,116, filed Sep. 26, 2022, entitled “Methods and Systems for Full Gene Length Single Point Mutagenesis,” by Weitz, et al., and U.S. Provisional Patent Application Ser. No. 63/410,140, filed Sep. 26, 2022, entitled “Systems and Methods for Screening of Large Gene Libraries,” by Weitz, et al. Each of these is incorporated herein by reference in its entirety.

### TECHNICAL FIELD

Mutagenesis, including single point mutagenesis, for large library generation is generally described.

### BACKGROUND

There is an increasing demand for methods to construct nucleic acid libraries in an efficient and scalable way. Error-prone PCR (epPCR), where mutations are randomly induced with a high mutation rate polymerase during thermal cycling, remains one of the most used technologies to prepare random mutagenesis libraries since it is cheap, simple, and scalable. However, the limitations of epPCR are also significant, including introducing silent mutations that only change expression level but do not alter the phenotype. In addition, it is extremely unlikely to have multiple substitutions in a single codon, which limits the possible searching space.

### SUMMARY

Systems and methods, and associated compositions, for producing large libraries of nucleic acids are generally described. The methods provided herein may be used, for example, to produce libraries that include mutations that are unlikely in conventional library generation techniques, and may facilitate the production of large libraries with reduced processing demands. The subject matter of the present disclosure involves, in some cases, interrelated products, alternative solutions to a particular problem, and/or a plurality of different uses of one or more systems and/or articles.

According to one aspect, a method of synthesizing a library of nucleic acid sequences. In some embodiments, the method comprises: synthesizing a first plurality of nucleic acids using a common template nucleic acid with a first plurality of primers; and synthesizing a second plurality of nucleic acids using one or more nucleic acids of the first plurality of nucleic acids; wherein the primers of the first plurality of primers define a first common template primer such that greater than or equal to 90% of the primers of the first plurality of primers differs from the first common template primer at exactly one codon; wherein the primers of the second plurality of primers define a second common template primer such that greater than or equal to 90% of the primers of the second plurality of primers differs from the second common template primer at exactly one codon, and wherein at least 5 consecutive nucleotides of the common template nucleic acid are complimentary to at least 5 consecutive nucleotides of the first common template primer.

In one aspect, a method of synthesizing a library of nucleic acid sequences is provided. In some embodiments, the method comprises: synthesizing a plurality of nucleic acids using a common template nucleic acid with a first plurality of primers and a second plurality of primers; wherein the primers of the first plurality of primers define a first common template primer such that greater than or equal to 90% of the primers of the first plurality of primers differs from the first common template primer at exactly one codon; wherein the primers of the second plurality of primers define a second common template primer such that greater than or equal to 90% of the primers of the second plurality of primers differs from the second common template primer at exactly one codon; and wherein at least 5 consecutive nucleotides of the common template nucleic acid are complimentary to at least 5 consecutive nucleotides of the first common template primer, and at least at least 5 consecutive nucleotides of the common template nucleic acid are complimentary to at least 5 consecutive nucleotides of the second common template primer, which are not complementary to the same portion of the common template nucleic acid as the first common template primer.

In another aspect, a method of synthesizing a library of nucleic acid sequences is provided. In some embodiments, the method comprises: synthesizing a first plurality of nucleic acids using a common template nucleic acid with a first plurality of primers; synthesizing a second plurality of nucleic acids using the common template nucleic acid with a second plurality of primers; and synthesizing a third plurality of nucleic acids using the first plurality of nucleic acids and the second plurality of nucleic acids, wherein the primers of the first plurality of primers defines a first common template primer such that greater than or equal to 90% of the primers of the first plurality of primers differs from the first common template primer at exactly one codon; and wherein the primers of the second plurality of primers defines a second common template primer such that greater than or equal to 90% of the primers of the second plurality of primers differs from the second common template primer at exactly one codon.

In yet another aspect, a solution is provided. According to some embodiments, the solution comprises: a plurality of nucleic acids, wherein greater than or equal to 50% of the nucleic acids each differs from a common template nucleic acid at at least one codon; and a plurality of primers, wherein greater than or equal to 50% of the primers of the plurality of primers differ from a common template primer at exactly one codon, wherein at least 5 consecutive nucleotides of the common template nucleic acid is complimentary to at least 5 consecutive nucleotides of the common template primer; wherein at least 90% of the plurality of primers have a structure such that the exactly one codon of the primer is not complementary to a sequentially corresponding codon of the common template nucleic acid.

In another aspect, a method of synthesizing a library of nucleic acid sequences is provided. According to some embodiments, the method comprises: synthesizing a first plurality of nucleic acids using a common template nucleic acid with a first plurality of primers; and synthesizing a second plurality of nucleic acids using one or more nucleic acids of the first plurality of nucleic acids; wherein the primers of the first plurality of primers define a first common template primer such that greater than or equal to 90% of the primers of the first plurality of primers differs from the first common template primer at greater than or equal to 1 and less than or equal to 4 codons; and wherein at least 5 consecutive nucleotides of the common template nucleic acid are complimentary to at least 5 consecutive nucleotides of the first common template primer.

In one aspect, a method of synthesizing a library of nucleic acid sequences is provided. According to some embodiments, the method comprises: synthesizing a plurality of nucleic acids using a common template nucleic acid with a first plurality of primers and a second plurality of primers; wherein the primers of the first plurality of primers define a first common template primer such that greater than or equal to 90% of the primers of the first plurality of primers differs from the first common template primer at greater than or equal to 1 and less than or equal to 4 codons; wherein the primers of the second plurality of primers define a second common template primer such that greater than or equal to 90% of the primers of the second plurality of primers differs from the second common template primer at at greater than or equal to 1 and less than or equal to 4 codons; and wherein at least 5 consecutive nucleotides of the common template nucleic acid are complimentary to at least 5 consecutive nucleotides of the first common template primer, and at least at least 5 consecutive nucleotides of the common template nucleic acid are complimentary to at least 5 consecutive nucleotides of the second common template primer, which are not complementary to the same portion of the common template nucleic acid as the first common template primer.

In another embodiment, a method of synthesizing a library of nucleic acid sequences is provided. Accorded to some embodiments, the method comprises: synthesizing a first plurality of nucleic acids using a common template nucleic acid with a first plurality of primers; synthesizing a second plurality of nucleic acids using the common template nucleic acid with a second plurality of primers; and synthesizing a third plurality of nucleic acids using the first plurality of nucleic acids and the second plurality of nucleic acids, wherein the primers of the first plurality of primers defines a first common template primer such that greater than or equal to 90% of the primers of the first plurality of primers differs from the first common template primer at at greater than or equal to 1 and less than or equal to 4 codons; and wherein the primers of the second plurality of primers defines a second common template primer such that greater than or equal to 90% of the primers of the second plurality of primers differs from the second common template primer at greater than or equal to 1 and less than or equal to 4 codons.

In yet another embodiment, a solution is provided. According to some embodiments, the solution comprises: a plurality of nucleic acids, wherein greater than or equal to 50% of the nucleic acids each differs from a common template nucleic acid at at least one codon; and a plurality of primers, wherein greater than or equal to 50% of the primers of the plurality of primers differ from a common template primer at greater than or equal to 1 and less than or equal to 4 codons, wherein at least 5 consecutive nucleotides of the common template nucleic acid are complimentary to at least 5 consecutive nucleotides of the common template primer; wherein at least 90% of the plurality of primers have a structure such that the at greater than or equal to 1 and less than or equal to 4 codons of the primer are not complementary to a sequentially corresponding codons of the common template nucleic acid.

Other advantages and novel features of the present disclosure will become apparent from the following detailed description of various non-limiting embodiments of the disclosure when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present disclosure will be described by way of example with reference to the accompanying figures, which are schematic and are not intended to be drawn to scale unless otherwise indicated. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIG. 1 presents a schematic illustration of a circular nucleic acid and a plurality of primers suitable for preparing a nucleic acid library, according to some embodiments;

FIG. 2A presents a schematic illustration of a nucleic acid and pluralities of primers, according to some embodiments;

FIG. 2B presents a schematic illustration of a nucleic acid and pluralities of primers, according to some embodiments;

FIG. 2C presents a schematic illustration of a nucleic acid and pluralities of primers, according to some embodiments;

FIG. 2D presents a schematic illustration of nucleic acids representing libraries of nucleic acids, according to some embodiments;

FIG. 2E presents a schematic illustration of nucleic acids representing a library of nucleic acids, according to some embodiments;

FIG. 3 presents a comparison of activities of enzymes produced by a method herein, according to some embodiments;

FIGS. 4A-4B show the mutation frequencies of a non-limiting library of nucleic acid sequences, according to some embodiments;

FIGS. 5A-5B show the mutation frequencies of a non-limiting library of nucleic acid sequences, according to some embodiments;

FIGS. 6A-6B show the mutation frequencies of a non-limiting library of nucleic acid sequences, according to some embodiments;

FIGS. 7A-7B show the mutation frequencies of a non-limiting library of nucleic acid sequences, according to some embodiments; and

FIGS. 8A-8B show the mutation frequencies of a non-limiting library of nucleic acid sequences, according to some embodiments.

### DETAILED DESCRIPTION

Library based methods using large libraries of biomolecules are important, modern tools that are useful in any of a variety of applications. One aspect of performing a library based method is starting with a robust library of biomolecules such as nucleic acids. One approach for generating biomolecular libraries is by mutating a template molecule (e.g., a template nucleic acid) through a process such as error-prone PCR. However, error-prone PCR is nonrandom; certain codon mutations are more likely than others, and some codon mutations are forbidden. Using the systems and methods described herein, certain libraries may be generated by a more uniformly random process, without any forbidden mutations, in some embodiments. The systems and methods described herein may thereby surpass the performance of conventional methods of library generation in some cases, such as error-prone PCR, by producing more functionally robust and diverse biomolecular libraries, with higher randomness, using fewer steps.

To provide a nonlimiting example, introduced solely for illustrative purposes, according to some embodiments, one embodiment comprises an iterative process of synthesizing a plurality of nucleic acids that differ from a common template nucleic acid at exactly one codon using PCR. Mutation of the nucleic acid may be assured by using a plurality of primers that each differs from a first common template primer at exactly one codon, where the common template primer perfectly complements a first portion of the common template nucleic acid. The plurality of primers may be chosen to include every possible codon mutation. For example, if a common template primer were 10 codons long, the plurality of primers could be chosen to include 190 primers, such that each codon of the common template primer is replaced by a codon expressing a different amino acid (as there are 20 naturally-occurring amino acids, there would be 19 possible replacements for each codon). Alternatively, in some embodiments, only a subset of possible primers is used.

The plurality of nucleic acids that differ from the common template nucleic acid at exactly one codon can be synthesized, for example, using PCR. One or more nucleic acids of the plurality of nucleic acids may be mutated using a second plurality of primers also present in the solution, where each of the primers of the second plurality of primers differs from a second common template primer at exactly one codon. In some cases, the second common template primer may perfectly complement a second portion of the common template nucleic acid. The second plurality of primers can introduce a second mutation corresponding to the second portion of the common template nucleic acid, producing nucleic acids differing from the common template nucleic acid by exactly two codons.

The likelihood of a given mutation may be controlled by the concentration of the primers, and thus uniform randomness of point mutations may be achieved by selecting appropriate primer concentrations (typically, for example, equimolar concentrations will result in about random mutation frequency). By iterating this process, robust libraries may be generated, including mutations that are highly improbable using more conventional techniques such as error-prone PCR. Of course, it should be understood that this example represents a specific and nonlimiting embodiment of the more general methods described below. For example, in some embodiments, two, three, four, or more mutations may be introduced in a primer, e.g., as discussed herein.

Thus, in one aspect, systems and methods of synthesizing a library of nucleic acid sequences are provided. In some cases, a common template nucleic acid and a first plurality of primers may be used to synthesize a first plurality of nucleic acid sequences.

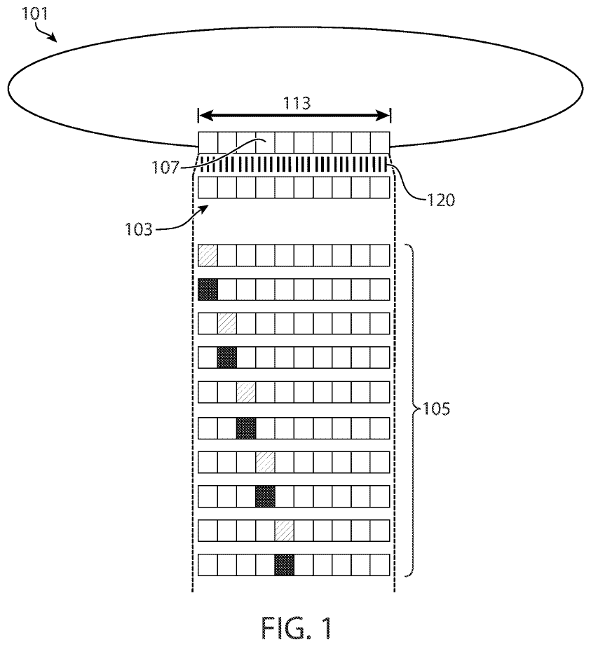

In some embodiments, it may be advantageous to use a circular nucleic acid as a common template nucleic acid. However, linear nucleic acids may also be used in other embodiments. FIG. 1 provides a schematic illustration of a non-limiting common template nucleic acid, with nucleic acid 101 represented as a circular nucleic acid. The white rectangles of nucleic acid 101 represent individual codons of nucleic acid 101 that are illustrated for emphasis—but the black curve is also part of nucleic acid 101 that is not shown in detail.

The common template nucleic acid may be selected by a user, and is not limiting. Any suitable template nucleic acid can be used. For example, a template nucleic acid may be chosen because it expresses a protein or other target of interest, of which a favorable improvement is desired. For instance, the template nucleic acid may express an enzyme capable of performing a useful biological or chemical function. In some embodiments, the common template nucleic acid is a circular nucleic acid. In certain embodiments, circular nucleic acids are used, which may be advantageous in some cases because only forward primers need be used. However, the techniques provided herein are not so limited, and noncircular nucleic acids may be used in other embodiments.

In some cases, certain primers are contained within a solution to promote amplification of a nucleic acid. The method may comprise using a plurality of primers to synthesize a plurality of nucleic acids using the common template nucleic acid. For example, FIG. 1 shows a plurality of primers 105 that are configured to synthesize a plurality of nucleic acids using nucleic acid 101.

In certain embodiments, a plurality of different types of primers may be added to the solution. At least some primers of the plurality of primers may comprise a plurality of consecutive nucleotides that are complementary to a plurality of consecutive nucleotides of the common template nucleic acid. In this way, a primer may be configured to bind to the common template nucleic acid, such that the primer can be used to synthesize a nucleic acid by an amplification technique (e.g., PCR), as described in greater detail below. At least some primers comprising a plurality of consecutive nucleotides complementary to nucleotides of the common template nucleic acid may include at least one codon that is not complementary to a codon of the template nucleic acid. In some embodiments, at least some primers comprising a plurality of consecutive nucleotides complementary to nucleotides of the common template nucleic acid include less than or equal to 4 codons (e.g., less than or equal to 3 codons, less than or equal to 2 codons) that are not complementary to codons of the common template nucleic acid. For example, the primer may include exactly one codon that is not complementary to a codon of the template nucleic acid.

Referring again to FIG. 1, plurality 105 of primers includes ten primers, each comprising 10 codons, represented schematically as individual rectangles. The nine white codons of each primer are complementary to portion 113 of nucleic acid 101; the shaded codons represent a codon that is not complementary to codon 107 of nucleic acid 101.

A primer may be configured to bind to a common template nucleic acid, according to some embodiments. For example, referring again to FIG. 1, each white codon of a primer of plurality of primers 105 includes 3 nucleotides that are complementary to 3 nucleotides of portion 113 of nucleic acid 101.

In some embodiments, greater than or equal to 5, greater than or equal to 10, greater than or equal to 15, greater than or equal to 20, greater than or equal to 25, greater than or equal to 30, or more consecutive nucleotides of the common template nucleic acid are complimentary to complementary nucleotides of the common template primer. In some embodiments, less than or equal to 100, less than or equal to 75, less than or equal to 50, less than or equal to 40, less than or equal to 30, less than or equal to 25, or fewer consecutive nucleotides of the common template nucleic acid are complimentary to complementary nucleotides of the common template primer. Combinations of these ranges are possible. For example, in some embodiments, greater than or equal to 5 and less than or equal to 100 consecutive nucleotides of the common template nucleic acid are complimentary to complementary nucleotides of the common template primer. Other ranges are also possible.

The primers may be present within the solution at any suitable density. For example, the primers may have a density of greater than or equal to 0.1 micromolar, greater than or equal to 0.3 micromolar, greater than or equal to 0.5 micromolar, greater than or equal to 0.8 micromolar, greater than or equal to 1 micromolar, greater than or equal to 5 micromolar, or more. In some embodiments, the primers have a density of less than or equal to 100 micromolar, less than or equal to 50 micromolar, less than or equal to 20 micromolar, less than or equal to 10 micromolar, less than or equal to 5 micromolar, less than or equal to 1 micromolar, or less. Combinations of these ranges are also possible (e.g., greater than or equal to 0.1 micromolar and less than or equal to 100 micromolar). Other ranges are also possible. The density may be independent of the density of nucleic acids. In some cases, an excess of primers is used, e.g., such that nucleic acids to be amplified by the primer control the reaction.

Any of a variety of suitable numbers of primers may be used. For instance, referring again to the example of FIG. 1, although 10 primers are shown in plurality 105 of primers, it should, of course, be understood that the plurality may include any appropriate number of primers, and 10 primers is shown by way of example only.

In some cases, at least 2, at least 3, at least 5, at least 10, at least 15, at least 20, at least 25, at least 30, at least 40, at least 50, at least 60, at least 75, at least 100, at least 150, at least 200, at least 500, at least 1,000, at least 2,000, at least 5,000, etc., different primers may be used. This may allow, for example, synthesis of a plurality of nucleic acids having one or more codons that differ from a common template nucleic acid. A variety of different nucleic acids may be amplified within different droplets.

The amplification may also be relatively selective, e.g., if library generation is centered on mutations of single, common template nucleic acid, by providing only certain primers. For instance, one or only a relatively small number of primers (e.g., less than or equal to 500, 400, 300, 200, 180, 160, 140, 120, 100, 80, 60, 40, 20, 19, 18, 17, 16, 15, 14, 13, 12, 11, 10, 9, 8, 7, 6, 5, 4, or 3 primers) may be provided in certain embodiments, thereby allowing only specific nucleic acid sequences to be amplified, e.g., within the droplets. In some cases, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 40, at least 60, at least 80, at least 100, at least 120, at least 140, at least 160, at least 180, or at least 200 primers may be present. Combinations of the aforementioned ranges (e.g., at least 2 and less than or equal to 500) are also possible. In some embodiments, exactly one primer is used e.g., for the purpose of amplifying, rather than mutating, circular nucleic acids present in the solution.

As a non-limiting example, primers that allow only certain mutations in a nucleic acid to be amplified may be used during amplification. For instance, a plurality of primers may be used that have relatively small differences, e.g., such that the primers have at least 70%, at least 75%, at least 80%, at least 85%, at least 90%, or at least 95% homology, and/or such that the amplification primers are all substantially complementary to a common template primer, except for no more than 12, 11, 10, 9, 8, 7, 6, 5, 4, 3, 2, or 1 nucleotide differences. For example, referring again to FIG. 1, each primer of plurality 105 of primers includes exactly one non-complementary codon (including no more than 3, 2, or 1 nucleotide differences). However, in other embodiments, more than one non-complementary codon may be present in a primer.

In some embodiments, amplification using multiple pluralities of primers can be used to produce a plurality of nucleic acids. For instance, in certain embodiments, the plurality of primers may represent variations on a common template primer, that is perfectly complementary to a portion of the nucleic acid. As a non-limiting example, in addition to plurality of primers 105, FIG. 1 shows common template primer 103 that is perfectly complementary to portion 113 of nucleic acid 101, as illustrated by nucleotide bonds 120 between portion 113 and common template primer 103.

In some embodiments, greater than or equal to 50%, greater than or equal to 60%, greater than or equal to 70%, greater than or equal to 80%, greater than or equal to 90%, greater than or equal to 95%, or more primers of a plurality differ from a common template primer at exactly one codon. In some embodiments, less than or equal to 100%, less than or equal to 95%, or fewer primers of a plurality differ from a common template primer at exactly one codon. Combinations of these ranges are possible. For example, in some embodiments, greater than or equal to 50% and less than or equal to 100% of primers of a plurality differ from a common template primer at exactly one codon. Other ranges are also possible.

In some embodiments, greater than or equal to 50%, greater than or equal to 60%, greater than or equal to 70%, greater than or equal to 80%, greater than or equal to 90%, greater than or equal to 95%, or more primers of a plurality differ from a common template primer at greater than or equal to 1 and less than or equal to 2 codons (e.g., at exactly 2 codons). In some embodiments, less than or equal to 100%, less than or equal to 95%, or fewer primers of a plurality differ from a common template primer at greater than or equal to 1 and less than or equal to 2 codons (e.g., at exactly 2 codons). Combinations of these ranges are possible. For example, in some embodiments, greater than or equal to 50% and less than or equal to 100% of primers of a plurality differ from a common template primer at greater than or equal to 1 and less than or equal to 2 codons (e.g., at exactly 2 codons). Other ranges are also possible. In addition, in some cases, greater than or equal to 50%, greater than or equal to 60%, greater than or equal to 70%, greater than or equal to 80%, greater than or equal to 90%, greater than or equal to 95%, or more primers of a plurality differ from a common template primer at greater than or equal to 1 and less than or equal to 4 codons or less than or equal to 3 codons.

In some embodiments, greater than or equal to 50%, greater than or equal to 60%, greater than or equal to 70%, greater than or equal to 80%, greater than or equal to 90%, greater than or equal to 95%, or more primers of a plurality differ from all other primers of the plurality at no more than two codons. In some embodiments, less than or equal to 100%, less than or equal to 95%, or fewer primers of a plurality differ from all other primers of the plurality at no more than two codons. Combinations of these ranges are possible. For example, in some embodiments, greater than or equal to 50% and less than or equal to 100% of primers of a plurality differ from all other primers of the plurality at no more than two codons. Other ranges are also possible. In addition, in some embodiments, greater than or equal to 50%, greater than or equal to 60%, greater than or equal to 70%, greater than or equal to 80%, greater than or equal to 90%, greater than or equal to 95%, or more primers of a plurality differ from all other primers of the plurality at no more than four codons or no more than three codons.

Amplification using a plurality of primers including exactly one codon that is not complementary to the common template nucleic acid (or two, three, or four codons) may thus produce a plurality of nucleic acids that differ from a common template nucleic acid. If the non-complementary codons of the primers occur in the same sequential position, amplification using the primers can produce a plurality of nucleic acids, each of which differs from the template nucleic acid at the non-complementary codon of the primer. Such a reaction may permit development of a library of nucleic acids via point-wise mutations, which may be particularly advantageous for library development, in some embodiments. As a non-limiting example, the common template nucleic acid may be amplified using 19 primers, each of which is non-complementary to the same codon, in order to produce a library of at least 19 nucleic acids (optionally including the common template nucleic acid, and additionally including 19 additional nucleic acids with a one-codon mutation) each of which would be identical, except for the changed codon. This can also readily be generalized to embodiment using two, three, or four codon mutations, etc.

More generally, a plurality of nucleic acids may comprise 19 primers for each codon of a common template primer (based on the 20 naturally-occurring amino acids) in certain embodiments as discussed herein. For example, if a common template primer included 10 codons, the plurality could include up to 190 primers, reflecting every possible single-codon variation of the common template primer. However, in some cases, more or fewer than 19 primers for each codon may be used, for example, to include non-naturally-occurring amino acids or to omit certain amino acids, etc.

An advantageous feature of this approach, in some embodiments, is that a primer differing from the common template nucleic acid at exactly one codon can be amplified either using the common template nucleic acid or using a previously-synthesized nucleic acid. Even if (as is highly probable) a first primer of the plurality was amplified from a nucleic acid produced using second primer of the plurality, the mutation of the second primer would not be incorporated into the synthesized nucleic acid, since both primers have the same length and correspond to the same template primer.

Thus, the number of unique nucleic acids included in the library may be controlled by the primers used, and generally cannot produce unexpected mutations. Another advantage of this approach is that the library may be used in some embodiments to encode any or every natural amino acid at a given site, and there are no forbidden mutations. Yet another advantage is that mutations known to be detrimental may be deliberately excluded in certain embodiments, since the mutation process may be controlled, at least in part, by the primers used to amplify the nucleic acids.

Multiple mutations can be made simultaneously in some embodiments. For example, two or more pluralities of primers could be used simultaneously in some cases. Primers within a first plurality may be configured to bind to a first portion of the common template nucleic acid (e.g., by corresponding to a first common template primer) and primers within a second plurality may be configured to bind to a second portion of the common template nucleic acid (e.g., by corresponding to a second common template primer, distinct from the first).

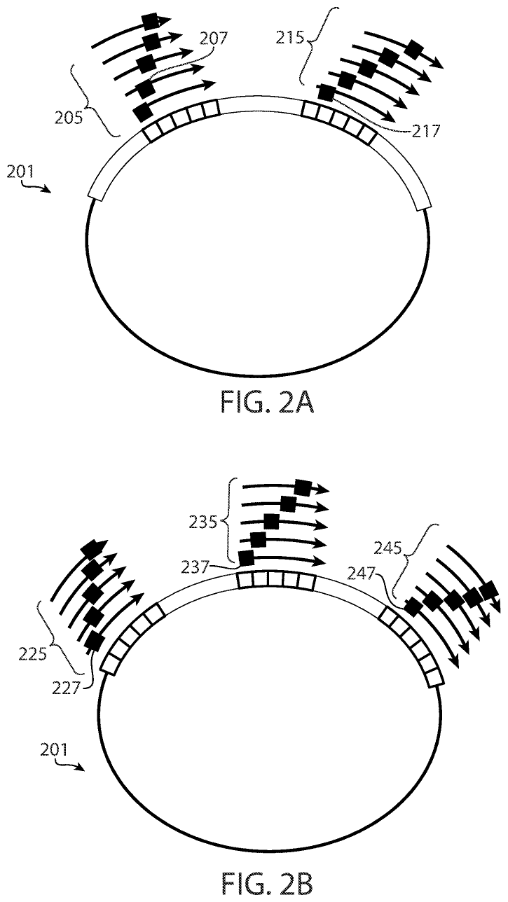

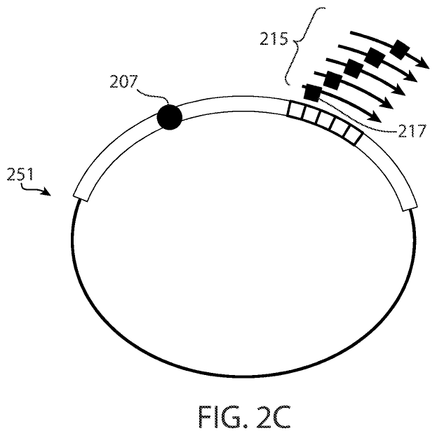

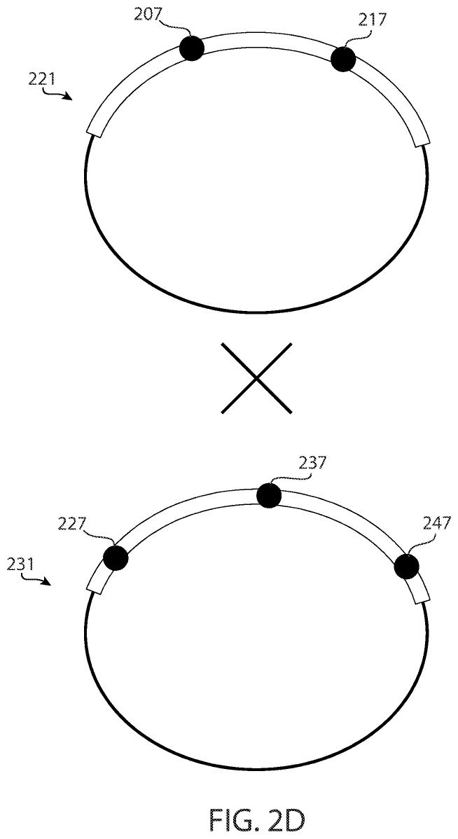

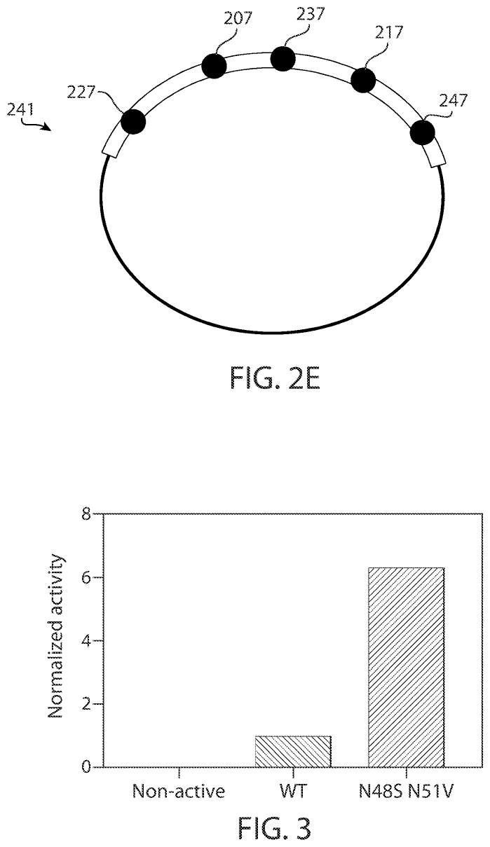

FIG. 2A presents an exemplary, schematic illustration of a non-limiting embodiment, where first plurality of primers 205 are complementary to the adjacent portion of nucleic acid 201, except at codons 207 indicated by black rectangles. In FIG. 2A, second plurality of primers 215 is complementary to nucleic acid 201 except at codons 217 indicated by black rectangles. Although the pluralities 205 and 215 are represented as including only five primers, this this number was chosen arbitrarily (for explanatory purposes only) and each primer may, in practice, include any of a variety of appropriate numbers of primers, as discussed above.

In some embodiments, the first portion of the common template nucleic acid and the second portion of the common template nucleic acid do not overlap. For example, referring again to FIG. 2A, first plurality of primers 205 does not complement the same portion of nucleic acid 201 as second plurality of primers 215.

However, in some embodiments, the first portion of the common template nucleic acid and the second portion of the common template nucleic acid may at least partially overlap, as the disclosure is not so limited. Within each plurality of primers, at least some (e.g., all) of the primers may be non-complementary to the common template nucleic acid at exactly one codon. For example, referring to FIG. 2A, each primer of plurality of primers 205 is non-complementary to nucleic acid 201 only at codon 207 and plurality of primers 215 is non-complementary to nucleic acid 201 only at codon 217.

Upon amplification, the first plurality of primers can be used to produce a mutation at a first sequential position of a common template nucleic acid and the second plurality of primers can be used to produce a mutation at a second sequential position of the common template nucleic acid, different from the first sequential position. Thus, two pluralities of primers may be used to produce a library including every pairwise combination of the first mutation and the second mutation, producing a library including up to 20n×20m distinct nucleic acid sequences, where “n” is the length of the primers of the first plurality of primers, and “m” is the length of the second plurality of primers. The lengths may be independent of each other. In this way, a library of nucleic acids may be designed that differs from a common template nucleic acid at up to 1, up to 2, up to 3, up to 4, up to 5, up to 6, up to 7, up to 8, up to 9, up to 10, up to 15, up to 20, up to 25, up to 30, up to 35, up to 40, up to 45, up to 50, up to 60, up to 70, up to 80, up to 90, up to 100, up to 150, up to 200, up to 250, up to 300, up to 400, up to 500, up to 1,000 or more codons, by using an appropriate number of primers differing at exactly one codon from the template nucleic acid.

In some embodiments, separate pools of primers may be used to mutate different portions of the common template nucleic acid. For example, FIG. 2A may represent the contents of a first pool, including common template nucleic acid 201, first plurality of primers 205, and second plurality of primers 215, while FIG. 2B represents a second pool, including the common template nucleic acid 201, third plurality 225 of primers complementary to nucleic acid 201 except at one of codons 227, fourth plurality 235 of primers complementary to nucleic acid 201 except at one of codons 237, and fifth plurality 245 of primers complementary to nucleic acid 201 except at one of codons 247.

In some embodiments, techniques such as the above example may produce a plurality of nucleic acids in a first pool, each differing from the common template nucleic acid at at least one codon, and a plurality of the nucleic acids in the first pool, each differing from the common template nucleic acid at at least one codon in a second pool. Each of these pools would contain a plurality of nucleic acids differing from the common template nucleic acid by exactly one codon. The use of different pools may be advantageous, in some embodiments, since it may permit point mutations of nearby codons without using overlapping primers in the same pool.

As discussed above, one or more nucleic acids of a plurality of nucleic acids prepared using a first plurality of common template primers may be mutated using a second plurality of primers also present in the solution. For example, FIG. 2C presents a nucleic acid 251, representative of a plurality of nucleic acids formed by amplification of nucleic acid 201 with first plurality of primers 205 as shown in FIG. 2A. Codons 207, mutated by plurality of primers 205 shown in FIG. 2A, are represented with a black circle representing any arbitrary mutation that could result from first plurality of primers 205 shown in FIG. 2A. Nucleic acids 251 of the plurality of nucleic acids can then react with primers of the second plurality of primers 215, where each of the primers of second plurality of primers 215 differs from a second common template primer at exactly one codon. Second plurality of primers 215 can introduce a second mutation corresponding to the second portion of the common template nucleic acid, producing nucleic acids differing from the common template nucleic acid by exactly two codons.

Although these figures only directly pertain to pluralities of primers that differ from the common template primer at exactly one codon, it should, of course, be understood that other embodiments are also possible. For example, in some embodiments a plurality of primers includes primers that differ from the common template primer at two codons, depending on the embodiments. The use of primers differing at more than two codons may be used, in some embodiments, to increase the mutation rate.

In some embodiments, a first plurality of nucleic acids and a second plurality of nucleic acids may be used to synthesize a third plurality of nucleic acids, differing from the common template nucleic acid at more than one codon. For example, in some embodiments the first plurality of nucleic acids and the second plurality of nucleic acids may be mixed and shuffled using any of a variety of methods known to those of ordinary skill in the art. The mixing and amplifying may be used to produce at third plurality of nucleic acids, wherein the third plurality of nucleic acids is identical to the common template nucleic acid except where the first plurality of nucleic acids or the second plurality of nucleic acids differs from the common template nucleic acid. For example, the third plurality of nucleic acids may be produced using any of a variety of suitable techniques, such as nucleic acid shuffling (e.g., DNA shuffling), overlap amplification (e.g., overlap PCR), or staggered extension (e.g., a process comprising or consisting of priming a nucleic acid of the plurality, followed by repeated cycles of denaturation and very short annealing and polymerase-catalyzed extension, wherein denaturation allows the partially-extended chains to melt away from their initial nucleic acids and bind to other nucleic acids, prior to a subsequent extension step).

In some embodiments, the third plurality of nucleic acids is the library of nucleic acids. It should, of course, be understood that one or more additional steps of using an additional plurality of primers to synthesize an additional plurality of nucleic acids and/or of performing additional techniques such as DNA shuffling, overlap amplification, or staggered extension may also be performed, and that the steps of synthesis may be performed in any of a variety of appropriate orders, depending on the embodiment. The use of additional steps may contribute to the formation of even larger libraries.

FIGS. 2D-2E present a non-limiting example of formation of a third plurality of nucleic acids. In FIG. 2D, a first plurality of nucleic acids 221, which would be formed by the pool of FIG. 2A, and a second plurality of nucleic acids 231, which would be formed by the pool of FIG. 2B, are pooled together. Only one nucleic acid is shown for each plurality, but it represents a generic structure of nucleic acids of each plurality of nucleic acids. Codons 207, 217, 227, 237, and 247, mutated by pluralities 205, 215, 225, 235, and 245 of primers shown in FIGS. 2A-2B, are represented with black circles, each representing any arbitrary mutation that could result from the associated pluralities of primers shown in FIGS. 2A-2B. FIG. 2E represents the plurality nucleic acids 241 that would result from mixing and amplifying the pluralities of nucleic acids 221 and 231 shown in FIG. 2D. Nucleic acids 241 are identical to the common template nucleic acid, except where the nucleic acids 221 or nucleic acids 231 differ from the common template nucleic acid 201 shown in FIGS. 2A-2B.

Libraries (e.g., third pluralities of nucleic acids) provided herein may be of any of a variety of appropriate sizes. In some embodiments, a library of nucleic acid sequences produced by a method provided herein comprises greater than or equal to 10⁵, greater than or equal to 10⁶, greater than or equal to 10⁷, greater than or equal to 10⁸, greater than or equal to 10⁹, greater than or equal to 10¹⁰, greater than or equal to 10¹¹, greater than or equal to 10¹², greater than or equal to 10¹³ or more sequences. According to some embodiments, a library of nucleic acid sequences produced by a method provided herein comprises less than or equal to 10¹⁴, less than or equal to 10¹³, less than or equal to 10¹², less than or equal to 10¹¹, less than or equal to 10¹⁰, less than or equal to 10⁹, less than or equal to 10⁸, or less sequences. Combinations of these ranges are also possible. For example, in some embodiments a library of nucleic acids comprises greater than or equal to 10⁵ and less than or equal to 10¹⁴ sequences.

The methods provided herein may provide any of a variety of suitable rates of mutations per round. Notably, high mutation rates per round may be advantageous for preparing libraries as described herein, since high mutation rates may allow the preparation of larger nucleotide libraries with fewer rounds of mutation. In some embodiments, a method provided herein includes a step of synthesizing a plurality of nucleic acids with a maximum mutation rate less than or equal to 32 mutations per kb per round, less than or equal to 30 mutations per kb per round, less than or equal to 25 mutations per kb per round, less than or equal to 22 mutations per kb per round, less than or equal to 20 mutations per kb per round, less than or equal to 15 mutations per kb per round, less than or equal to 12 mutations per kb per round, less than or equal to 10 mutations per kb per round, less than or equal to 5 mutations per kb per round, less than or equal to 2 mutations per kb per round, or less. In some embodiments, a method provided herein includes a step of synthesizing a plurality of nucleic acids with a maximum mutation rate of greater than or equal to 1 mutations per kb per round, greater than or equal to 2 mutations per kb per round, greater than or equal to 5 mutations per kb per round, greater than or equal to 10 mutations per kb per round, greater than or equal to 12 mutations per kb per round, greater than or equal to 15 mutations per kb per round, greater than or equal to 20 mutations per kb per round, greater than or equal to 22 mutations per kb per round, greater than or equal to 25 mutations per kb per round, greater than or equal to 30 mutations per kb per round, or greater. Combinations of these ranges are also possible. For example, in some embodiments, a method provided herein includes a step of synthesizing a plurality of nucleic acids with a maximum mutation rate of greater than or equal to 1 mutation per kb per round and less than or equal to 32 mutations per kb per round.

Another advantage of methods provided herein is that mutagenesis performed using a plurality of primers provided herein may have a very low or negligible likelihood of causing a frame-shift during mutagenesis. This may result in the preparation of libraries with relatively few early stop-codons, even when libraries are prepared using a high mutation rate as discussed above. Libraries (e.g., third pluralities of nucleic acids) provided herein include a very small proportion of sequences with early stop codons. In some embodiments, less than or equal to 10%, less than or equal to 5%, less than or equal to 2.5%, less than or equal to 1% or less of the sequences of the library comprise an early stop codon. According to some embodiments, greater than or equal to 0.1%, greater than or equal to 0.2%, greater than or equal to 0.5% or greater than or equal to 1% of the sequences of the library comprise early stop codons. Combinations of these ranges are also possible. For example, in some embodiments a library of nucleic acids produced according to a method provided herein includes early stop codons in greater than or equal to 0.1% and less than or equal to 10 wt % of its sequences.

In some embodiments, sequential amplification steps may be used to produce a library. For example, a first plurality of nucleic acids may be synthesized, e.g., as described above. Then, at least some nucleic acids of the first plurality of nucleic acids may be amplified using a second plurality of primers to synthesize a second plurality of nucleic acids, e.g., as described above.

In some embodiments, certain systems and methods comprise determining a subset of the first plurality of nucleic acids that is associated with favorable mutations of the common template nucleic acid. Such a favorable subset may be identified by any of a variety of appropriate methods. For example, the subset may be identified by selecting for nucleic acids that bond to a target substrate. As another example, in some embodiments each of the nucleic acids are used to express a protein, and the activity of that protein is measured by an appropriate method (e.g., by detecting activity of a probe, such as a change in fluorescence of a fluorescent probe, or a color change of a colorometric indicator). For example, the protein may be mixed with an enzyme substrate, and enzymatic activity of the protein may be detected, as described in the examples below. The protein may be expressed by any of a variety of appropriate methods. For example, the protein may be expressed by performing a translation reaction to synthesize it, or by expressing the protein in a plasmid by inserting the nucleic acid into the plasmid. Other techniques are also possible, and the disclosure is not so limited.

Generally, any of a variety of appropriate numbers of nucleic acids of the first plurality of nucleic acids may be used to synthesize the second plurality of nucleic acids. For example, in some embodiments, greater than or equal to 1, greater than or equal to 2, greater than or equal to 5, greater than or equal to 10, greater than or equal to 20, greater than or equal to 50, or more nucleic acids of the first plurality may be used to synthesize the second plurality of nucleic acids.

In some embodiments, the less than or equal to 100%, less than or equal to 99%, less than or equal to 95%, less than or equal to 90%, less than or equal to 75%, less than or equal to 50%, or less of the nucleic acids of the first plurality may be used to synthesize the second plurality of nucleic acids. Combinations of the forgoing ranges are possible. For example, in some embodiments, at least one nucleic acid of the first plurality and less than or equal to 100% of the nucleic acids of the first plurality of nucleic acids may be used to synthesize the second plurality of nucleic acids. Other ranges are also possible. The nucleic acids of the first plurality used to synthesize the second plurality of nucleic acids may be collected by any of a variety of suitable methods. For example, the first plurality of nucleic acids may be broken into droplets, as discussed in greater detail below.

One advantage of sequential mutation is that the determination of the favorable subset may be performed between amplification steps, and a subset of a first plurality of nucleic acids that is associated with favorable mutations of the common template nucleic acid may be selected for amplification. For example, in some embodiments, a most favorable nucleic acid from the first plurality can be used to synthesize the second plurality of nucleic acids using the second plurality of primers. In some embodiments, the most favorable nucleic acid can be treated as a second common template nucleic acid, and primers may be designed to be complementary to the mutated codon, while introducing new mutations at the site of a different codon.

In some embodiments, some or all nucleic acids of the first plurality of nucleic acids are used to form the second plurality of nucleic acids. For example, the nucleic acids of the first plurality may be included in a single mixture, and may be amplified together within that mixture using the second plurality of primers. However, in some embodiments, the first plurality of nucleic acids may be partitioned, e.g., by breaking a mixture comprising the nucleic acids into a plurality of droplets and amplifying nucleic acids therein, as discussed below. Then, the second plurality of primers may be introduced into at least some of the plurality of droplets, in order to synthesize the second plurality of nucleic acids within the droplets.

Synthesis of the nucleic acids described herein may comprise, in some embodiments, an entirely sequential process of mutations, where one codon is mutated at a time. In some embodiments, the nucleic acids, one or more step may be used to introduce multiple mutations during the same reaction, as discussed above. Combinations of these steps may also be used. For example, in some embodiments, a method may comprise a first step, where 5 pluralities of primers are used to prepare a first plurality of nucleic acids, each differing by up to 5 codons from a common template nucleic acid. (Other numbers of primers may be used in other embodiments, for example, 2, 3, or 4, or 6, 7, 8, 9, 10, etc.; 5 is used by way of example only.) This method may further comprise using a sixth plurality of primers to introduce a mutation in exactly one additional codon of at least some nucleic acids of the first plurality. Intermediate steps, such as identifying a favorable subset of the first plurality of nucleic acids, may be used in concert with such a method. Such a method may be useful for identifying useful mutations of proteins that are already favorably mutated relative to a wild type protein, permitting a directed process of library evolution.

Generally, some of the systems and methods described herein may be used in any of a variety of suitable chemical environments. For example, some systems and methods may be performed in solution. The solution may be an aqueous solution, and may comprise any appropriate mixture of salts and/or buffers. In some embodiments, the solution may comprise one or more additional agents, such as a reagent for determining favorability of nucleic acid (e.g., an enzyme substrate), a reagent for amplifying a nucleic acid (e.g., a polymerase or a nucleic acid monomer such as T, A, C, G, or U), a probe (e.g. a fluorescent colorimetric probe), and/or a nonaqueous solvent such as ethanol or isopropanol. Other compounds may be included as well, and the disclosure is not so limited.

In some cases, nucleic acids described herein may be amplified. Amplifying nucleic acids may be useful for synthesizing mutated nucleic acids, as discussed above. For example, amplification may be used to synthesize a new plurality of nucleic acids from a common template nucleic acid and/or an old plurality of nucleic acids. However, in some embodiments, nucleic acids are amplified for other reasons. For example, nucleic acids may be amplified (e.g., using primers that perfectly complement a portion of the nucleic acids, and do not introduce mutations) to produce a larger number or concentration of nucleic acids, e.g., for subsequent analysis, sequencing, or the like.

Those of ordinary skill in the art will be familiar with various amplification methods that can be used, including, but not limited to, polymerase chain reaction (PCR), reverse transcriptase (RT) PCR amplification, in vitro transcription amplification (IVT), multiple displacement amplification (MDA), or quantitative real-time PCR (qPCR).

Primers may be present during formation of the droplets, and/or added to the droplets, e.g., after formation of the droplets. In some embodiments, primers may be included during formation of a bulk solution and/or added after formation of the bulk solution. It should be noted that the manner in which the primers are added may be the same or different from the manner in which the nucleic acids are added to the droplets. For instance, the primers may be present during formation of droplets of the solution, and/or added to the droplets after formation of the droplets. Those of ordinary skill in the art will be aware of systems and methods of adding solutions to droplets.

Amplification of nucleic acids according to the techniques described herein may be performed in bulk, or using droplet methods as discussed in greater detail below. In some cases, the primers and/or nucleic acids, may be contained within droplets using techniques such as those described herein.

In some cases, the plurality of primers may be divided into different groups such that the droplets are exposed to different primers, e.g., that are injected into the droplets. This may allow synthesis of different nucleic acids of the first plurality of nucleic acids in different droplets, which may be advantageous for subsequent functionality assessment. However, in other embodiments, the primers may be distributed differently, e.g., such that some or all of the primers are present in the same solution. Thus, in some embodiments, the primers may be distributed such that some or all of the droplets contains only a single primer in certain embodiments, because different groups of droplets are used, a plurality of different targets may still be determined for a pool of amplified nucleic acids.

A solution containing both a nucleic acid and an appropriate primer may be treated to cause amplification of the nucleic acid to occur. The amplification may occur with or without mutations, depending on whether primers include non-complementary codons. This may allow a large amount or concentration of nucleic acids to be produced, e.g., while controlling the sequential distribution of nucleic acids. In some cases, the primers are selected to allow substantially all, or only some, of the nucleic acids present to be amplified. Techniques for amplifying nucleic acids include PCR (polymerase chain reaction) or any of the other techniques described herein.

As examples, PCR (polymerase chain reaction) may be used to amplify nucleic acids, e.g., contained within droplets. Typically, in PCR reactions, the nucleic acids are heated (e.g., to a temperature of at least about 50° C., at least about 70° C., or least about 90° C. in some cases) to cause dissociation of the nucleic acids into single strands, and a heat-stable DNA polymerase (such as Taq polymerase) is used to amplify the nucleic acid. This process is often repeated multiple times to amplify the nucleic acids.

In one set of embodiments, PCR amplification may be performed within the droplets. For example, the droplets may contain a polymerase (such as Taq polymerase), and DNA nucleotides (deoxyribonucleotides), and the droplets may be processed (e.g., via repeated heated and cooling) to amplify the nucleic acid within the droplets. Suitable reagents for PCR or other amplification techniques, such as polymerases and/or deoxyribonucleotides, may be added to the droplets during their formation, and/or afterwards (e.g., via merger with droplets containing such reagents, and/or via direct injection of such reagents, e.g., contained within a fluid). Various techniques for droplet injection or merger of droplets will be known to those of ordinary skill in the art. See, e.g., U.S. Pat. Apl. Pub. No. 2012/0132288, incorporated herein by reference. In some embodiments, primers may be added to the droplets, or the primers may be present on one or more of the nucleic acids within the droplets. Those of ordinary skill in the art will be aware of suitable primers, many of which can be readily obtained commercially.

In one set of embodiments, at least some of the primers may be distinguished, for example, using distinguishable fluorescent tags, barcodes, or other suitable identification tags. Examples of barcodes that can be contained within droplets include, but are not limited to, those described in U.S. Pat. Apl. Pub. No. 2018/0304222 or Int. Pat. Apl. Pub. No. WO 2015/164212, each incorporated herein by reference.

The nucleic acids may be amplified to any suitable extent. The degree of amplification may be controlled, for example, by controlling factors such as the temperature, cycle time, or amount of enzyme and/or deoxyribonucleotides contained within the droplets. For instance, in some embodiments, a population of droplets may have at least about 50,000, at least about 100,000, at least about 150,000, at least about 200,000, at least about 250,000, at least about 300,000, at least about 400,000, at least about 500,000, at least about 750,000, at least about 1,000,000 or more molecules of the amplified nucleic acid per droplet.

Amplified nucleic acids may be contained within droplets, in accordance with certain embodiments, e.g., for subsequent or amplification. The droplets may be created using any suitable technique, such as those described herein, and the technique for creating these droplets may be the same or different than for the initial droplets. In some cases, the droplets may also be monodisperse, and/or have distributions or dimensions such as are described herein. The amplified nucleic acids may be contained within droplets using any suitable technique, e.g., during or after the droplets have been formed. Techniques for creating droplets and/or adding fluid to a droplet have been discussed herein.

In some cases, the amplified nucleic acids may be contained within droplets at relatively low densities. For example, the droplets may, on the average contain less 1 nucleic acid per droplet. For example, the average loading rate may be less than about 1 particle/droplet, less than about 0.9 nucleic acids/droplet, less than about 0.8 nucleic acids/droplet, less than about 0.7 nucleic acids/droplet, less than about 0.6 nucleic acids/droplet, less than about 0.5 nucleic acids/droplet, less than about 0.4 nucleic acids/droplet, less than about 0.3 nucleic acids/droplet, less than about 0.2 nucleic acids/droplet, less than about 0.1 nucleic acids/droplet, less than about 0.05 nucleic acids/droplet, less than about 0.03 nucleic acids/droplet, less than about 0.02 nucleic acids/droplet, or less than about 0.01 nucleic acids/droplet. In some cases, lower densities may be chosen to minimize the probability that a droplet will have two or more nucleic acids in it. Thus, for example, at least about 50%, at least about 60%, at least about 70%, at least about 80%, at least about 90%, at least about 95%, at least about 97%, at least about 98%, or at least about 99% of the droplets may contain either no target nucleic acid or only one such nucleic acid. In addition, in some cases, the loading densities may also be controlled such that at least a signification amount of the droplets contains a target nucleic acid. This may be useful, for example, to prevent too much inefficiency in loading, or subsequent operations, etc. For instance, at least about 50%, at least about 60%, at least about 70%, at least about 80%, or at least about 90% of the droplets may also contain at least one such nucleic acid.

In some embodiments, a plurality of different droplet makers may be used, each of which introduces a primer into the droplets as they are formed. Examples of droplet makers include channel junctions such as a T-junction, a Y-junction, a channel-within-a-channel junction, a cross (or “X”) junction, a flow-focusing junction, or the like. Other suitable examples of different droplet makers and techniques for forming droplets include any of those discussed herein. Examples of techniques for introducing primers after droplet formation include picoinjection or other methods such as those discussed in Int. Pat. Apl. Pub. No. WO 2010/151776, incorporated herein by reference, through fusion of the droplets with droplets containing primers, or the like.

In some embodiments, a method comprises purifying nucleic acids (e.g., a plurality of nucleic acids produced using a template nucleic acid as described above). Purification may be used, for example, to extract the nucleic acids from unwanted reagents used in earlier steps. For example, purification may be used to extract the nucleic acids from proteins transcribed therefrom. Any of a variety of appropriate techniques may be used to purify the nucleic acids. For example, the nucleic acids may be purified using any of a variety of suitable methods, such as column- or gel-based methods (including electrophoretic and centrifuge-based methods). For example, nucleic acids may be purified using a PCR clean-up kit.

In some embodiments, a method comprises sequencing one or more nucleic acids. For example, it may be advantageous to sequence at least some nucleic acids of a first plurality of nucleic acids, prior to using the first plurality of nucleic acids to synthesize a second plurality of nucleic acids. Examples of methods for determining and/or sequencing nucleic acids include, but are not limited to, chain-termination sequencing, sequencing-by-hybridization, Maxam-Gilbert sequencing, dye-terminator sequencing, chain-termination methods, Massively Parallel Signature Sequencing (Lynx Therapeutics), polony sequencing, pyrosequencing, sequencing by ligation, ion semiconductor sequencing, DNA nanoball sequencing, single-molecule real-time sequencing (e.g., Pacbio sequencing), nanopore sequencing, Sanger sequencing, digital RNA sequencing (“digital RNA-seq”), Illumina sequencing, capillary electrophoresis, etc. In some cases, a microarray, such as a DNA microarray, may be used, for example to determine or identify nucleic acids. Those of ordinary skill in the art will be aware of other techniques that can be used to determine and/or sequence nucleic acids, e.g., qualitatively and/or quantitatively.

One or more steps of the systems and methods described above may be performed using droplet-based methods. In some embodiments, a plurality of primers and/or a plurality of nucleic acids is comprised by a same droplet. According to some embodiments, a plurality of primers and/or a plurality of nucleic acids is comprised by a plurality of separate droplets. Additional details regarding systems and methods for manipulating droplets in a microfluidic system follow, in accordance with certain aspects. For example, various systems and methods for screening and/or sorting droplets are described in U.S. patent application Ser. No. 11/360,845, filed Feb. 23, 2006, entitled “Electronic Control of Fluidic Species,” by Link, et al., published as U.S. Patent Application Publication No. 2007/000342 on Jan. 4, 2007, incorporated herein by reference. As a non-limiting example, in some aspects, by applying (or removing) a first electric field (or a portion thereof), a droplet may be directed to a first region or channel; by applying (or removing) a second electric field to the device (or a portion thereof), the droplet may be directed to a second region or channel; by applying a third electric field to the device (or a portion thereof), the droplet may be directed to a third region or channel; etc., where the electric fields may differ in some way, for example, in intensity, direction, frequency, duration, etc.

As mentioned, certain embodiments comprise a droplet contained within a carrying fluid. For example, there may be a first phase forming droplets contained within a second phase, where the surface between the phases comprises one or more proteins. For example, the second phase may comprise oil or a hydrophobic fluid, while the first phase may comprise water or another hydrophilic fluid (or vice versa). It should be understood that a hydrophilic fluid is a fluid that is substantially miscible in water and does not show phase separation with water at equilibrium under ambient conditions (typically 25° C. and 1 atm). Examples of hydrophilic fluids include, but are not limited to, water and other aqueous solutions comprising water, such as cell or biological media, ethanol, salt solutions, saline, blood, etc. In some cases, the fluid is biocompatible.

Similarly, a hydrophobic fluid is one that is substantially immiscible in water and will show phase separation with water at equilibrium under ambient conditions. As previously discussed, the hydrophobic fluid is sometimes referred to by those of ordinary skill in the art as the “oil phase” or simply as an oil. Non-limiting examples of hydrophobic fluids include oils such as hydrocarbons oils, silicon oils, fluorocarbon oils, organic solvents, perfluorinated oils, perfluorocarbons such as perfluoropolyether, etc. Additional examples of potentially suitable hydrocarbons include, but are not limited to, light mineral oil (Sigma), kerosene (Fluka), hexadecane (Sigma), decane (Sigma), undecane (Sigma), dodecane (Sigma), octane (Sigma), cyclohexane (Sigma), hexane (Sigma), or the like. Non-limiting examples of potentially suitable silicone oils include 2 cst polydimethylsiloxane oil (Sigma). Non-limiting examples of fluorocarbon oils include FC3283 (3M), FC40 (3M), Krytox GPL (Dupont), etc. In addition, other hydrophobic entities may be contained within the hydrophobic fluid in some embodiments. Non-limiting examples of other hydrophobic entities include drugs, immunologic adjuvants, or the like.

Thus, the hydrophobic fluid may be present as a separate phase from the hydrophilic fluid. In some embodiments, the hydrophobic fluid may be present as a separate layer, although in other embodiments, the hydrophobic fluid may be present as individual fluidic droplets contained within a continuous hydrophilic fluid, e.g. suspended or dispersed within the hydrophilic fluid. This is often referred to as an oil/water emulsion. The droplets may be relatively monodisperse, or be present in a variety of different sizes, volumes, or average diameters. In some cases, the droplets may have an overall average diameter of less than about 1 mm, or other dimensions as discussed herein. In some cases, a surfactant may be used to stabilize the hydrophobic droplets within the hydrophilic liquid, for example, to prevent spontaneous coalescence of the droplets. Non-limiting examples of surfactants include those discussed in U.S. Pat. Apl. Pub. No. 2010/0105112, incorporated herein by reference. Other non-limiting examples of surfactants include Span80 (Sigma), Span80/Tween-20 (Sigma), Span80/Triton X-100 (Sigma), Abil EM90 (Degussa), Abil we09 (Degussa), polyglycerol polyricinoleate “PGPR90” (Danisco), Tween-85, 749 Fluid (Dow Corning), the ammonium carboxylate salt of Krytox 157 FSL (Dupont), the ammonium carboxylate salt of Krytox 157 FSM (Dupont), or the ammonium carboxylate salt of Krytox 157 FSH (Dupont). In addition, the surfactant may be, for example, a peptide surfactant, bovine serum albumin (BSA), or human serum albumin.

The droplets may have any suitable shape and/or size. In some cases, the droplets may be microfluidic, and/or have an average diameter of less than about 1 mm. For instance, the droplet may have an average diameter of less than about 1 mm, less than about 700 micrometers, less than about 500 micrometers, less than about 300 micrometers, less than about 100 micrometers, less than about 70 micrometers, less than about 50 micrometers, less than about 30 micrometers, less than about 10 micrometers, less than about 5 micrometers, less than about 3 micrometers, less than about 1 micrometer, etc. The average diameter may also be greater than about 1 micrometer, greater than about 3 micrometers, greater than about 5 micrometers, greater than about 7 micrometers, greater than about 10 micrometers, greater than about 30 micrometers, greater than about 50 micrometers, greater than about 70 micrometers, greater than about 100 micrometers, greater than about 300 micrometers, greater than about 500 micrometers, greater than about 700 micrometers, or greater than about 1 mm in some cases. Combinations of any of these are also possible; for example, the diameter of the droplet may be between about 1 mm and about 100 micrometers. The diameter of a droplet, in a non-spherical droplet, may be taken as the diameter of a perfect mathematical sphere having the same volume as the non-spherical droplet.

In some embodiments, the droplets may be of substantially the same shape and/or size (i.e., “monodisperse”), or of different shapes and/or sizes, depending on the particular application. In some cases, the droplets may have a homogenous distribution of cross-sectional diameters, i.e., in some embodiments, the droplets may have a distribution of average diameters such that no more than about 20%, no more than about 10%, or no more than about 5% of the droplets may have an average diameter greater than about 120% or less than about 80%, greater than about 115% or less than about 85%, greater than about 110% or less than about 90%, greater than about 105% or less than about 95%, greater than about 103% or less than about 97%, or greater than about 101% or less than about 99% of the average diameter of the microfluidic droplets. Some techniques for producing homogenous distributions of cross-sectional diameters of droplets are disclosed in International Patent Application No. PCT/US2004/010903, filed Apr. 9, 2004, entitled “Formation and Control of Fluidic Species,” by Link, et al., published as WO 2004/091763 on Oct. 28, 2004, incorporated herein by reference. In addition, in some instances, the coefficient of variation of the average diameter of the droplets may be less than or equal to about 20%, less than or equal to about 15%, less than or equal to about 10%, less than or equal to about 5%, less than or equal to about 3%, or less than or equal to about 1%. However, in other embodiments, the droplets may not necessarily be substantially monodisperse, and may instead exhibit a range of different diameters.

Those of ordinary skill in the art will be able to determine the average diameter of a population of droplets, for example, using laser light scattering or other known techniques. The droplets so formed can be spherical, or non-spherical in certain cases. The diameter of a droplet, in a non-spherical droplet, may be taken as the diameter of a perfect mathematical sphere having the same volume as the non-spherical droplet.

In some embodiments, one or more droplets may be created within a channel by creating an electric charge on a fluid surrounded by a liquid, which may cause the fluid to separate into individual droplets within the liquid. In some embodiments, an electric field may be applied to the fluid to cause droplet formation to occur. The fluid can be present as a series of individual charged and/or electrically inducible droplets within the liquid. Electric charge may be created in the fluid within the liquid using any suitable technique, for example, by placing the fluid within an electric field (which may be AC, DC, etc.), and/or causing a reaction to occur that causes the fluid to have an electric charge.

The electric field, in some embodiments, is generated from an electric field generator, i.e., a device or system able to create an electric field that can be applied to the fluid. The electric field generator may produce an AC field (i.e., one that varies periodically with respect to time, for example, sinusoidally, sawtooth, square, etc.), a DC field (i.e., one that is constant with respect to time), a pulsed field, etc. Techniques for producing a suitable electric field (which may be AC, DC, etc.) are known to those of ordinary skill in the art. For example, in one embodiment, an electric field is produced by applying voltage across a pair of electrodes, which may be positioned proximate a channel such that at least a portion of the electric field interacts with the channel. The electrodes can be fashioned from any suitable electrode material or materials known to those of ordinary skill in the art, including, but not limited to, silver, gold, copper, carbon, platinum, copper, tungsten, tin, cadmium, nickel, indium tin oxide (“ITO”), etc., as well as combinations thereof.

In another set of embodiments, droplets of fluid can be created from a fluid surrounded by a liquid within a channel by altering the channel dimensions in a manner that is able to induce the fluid to form individual droplets. The channel may, for example, be a channel that expands relative to the direction of flow, e.g., such that the fluid does not adhere to the channel walls and forms individual droplets instead, or a channel that narrows relative to the direction of flow, e.g., such that the fluid is forced to coalesce into individual droplets. In some cases, the channel dimensions may be altered with respect to time (for example, mechanically or electromechanically, pneumatically, etc.) in such a manner as to cause the formation of individual droplets to occur. For example, the channel may be mechanically contracted (“squeezed”) to cause droplet formation, or a fluid stream may be mechanically disrupted to cause droplet formation, for example, through the use of moving baffles, rotating blades, or the like.

Some embodiments generally relate to systems and methods for fusing or coalescing two or more droplets into one droplet, e.g., where the two or more droplets ordinarily are unable to fuse or coalesce, for example, due to composition, surface tension, droplet size, the presence or absence of surfactants, etc. In certain cases, the surface tension of the droplets, relative to the size of the droplets, may also prevent fusion or coalescence of the droplets from occurring.

As a non-limiting example, two droplets can be given opposite electric charges (i.e., positive and negative charges, not necessarily of the same magnitude), which can increase the electrical interaction of the two droplets such that fusion or coalescence of the droplets can occur due to their opposite electric charges. For instance, an electric field may be applied to the droplets, the droplets may be passed through a capacitor, a chemical reaction may cause the droplets to become charged, etc. The droplets, in some cases, may not be able to fuse even if a surfactant is applied to lower the surface tension of the droplets. However, if the droplets are electrically charged with opposite charges (which can be, but are not necessarily of, the same magnitude), the droplets may be able to fuse or coalesce. As another example, the droplets may not necessarily be given opposite electric charges (and, in some cases, may not be given any electric charge), and are fused through the use of dipoles induced in the droplets that causes the droplets to coalesce. Also, the two or more droplets allowed to coalesce are not necessarily required to meet “head-on.” Any angle of contact, so long as at least some fusion of the droplets initially occurs, is sufficient. See also, e.g., U.S. patent application Ser. No. 11/698,298, filed Jan. 24, 2007, entitled “Fluidic Droplet Coalescence,” by Ahn, et al., published as U.S. Patent Application Publication No. 2007/0195127 on Aug. 23, 2007, incorporated herein by reference in its entirety.

In one set of embodiments, a fluid may be injected into a droplet. The fluid may be microinjected into the droplet in some cases, e.g., using a microneedle or other such device. In other cases, the fluid may be injected directly into a droplet using a fluidic channel as the droplet comes into contact with the fluidic channel. Other techniques of fluid injection are disclosed in, e.g., International Patent Application No. PCT/US2010/040006, filed Jun. 25, 2010, entitled “Fluid Injection,” by Weitz, et al., published as WO 2010/151776 on Dec. 29, 2010; or International Patent Application No. PCT/US2009/006649, filed Dec. 18, 2009, entitled “Particle-Assisted Nucleic Acid Sequencing,” by Weitz, et al., published as WO 2010/080134 on Jul. 15, 2010, each incorporated herein by reference in its entirety.

The following documents are each incorporated herein by reference in its entirety for all purposes: Int. Pat. Apl. Pub. No. WO 2016/168584, entitled “Barcoding System for Gene Sequencing and Other Applications,” by Weitz et al.; Int. Pat. Apl. Pub. No. WO 2015/161223, entitled “Methods and Systems for Droplet Tagging and Amplification,” by Weitz, et al.; U.S. Pat. Apl. Ser. No. 61/980,541, entitled “Methods and Systems for Droplet Tagging and Amplification,” by Weitz, et al.; U.S. Pat. Apl. Ser. No. 61/981,123, entitled “Systems and Methods for Droplet Tagging,” by Bernstein, et al.; Int. Pat. Apl. Pub. No. WO 2004/091763, entitled “Formation and Control of Fluidic Species,” by Link et al.; Int. Pat. Apl. Pub. No. WO 2004/002627, entitled “Method and Apparatus for Fluid Dispersion,” by Stone et al.; Int. Pat. Apl. Pub. No. WO 2006/096571, entitled “Method and Apparatus for Forming Multiple Emulsions,” by Weitz et al.; Int. Pat. Apl. Pub. No. WO 2005/021151, entitled “Electronic Control of Fluidic Species,” by Link et al.; Int. Pat. Apl. Pub. No. WO 2011/056546, entitled “Droplet Creation Techniques,” by Weitz, et al.; Int. Pat. Apl. Pub. No. WO 2010/033200, entitled “Creation of Libraries of Droplets and Related Species,” by Weitz, et al.; U.S. Pat. Apl. Pub. No. 2012-0132288, entitled “Fluid Injection,” by Weitz, et al.; Int. Pat. Apl. Pub. No. WO 2008/109176, entitled “Assay And Other Reactions Involving Droplets,” by Agresti, et al.; and Int. Pat. Apl. Pub. No. WO 2010/151776, entitled “Fluid Injection,” by Weitz, et al.; and U.S. Pat. Apl. Ser. No. 62/072,944, entitled “Systems and Methods for Barcoding Nucleic Acids,” by Weitz, et al.

In addition, the following are incorporated herein by reference in their entireties: U.S. Pat. Apl. Ser. No. 61/981,123 filed Apr. 17, 2014; PCT Pat. Apl. Ser. No. PCT/US2015/026338, filed Apr. 17, 2015, entitled “Systems and Methods for Droplet Tagging”; U.S. Pat. Apl. Ser. No. 61/981,108 filed Apr. 17, 2014; U.S. Pat. Apl. Ser. No. 62/072,944, filed Oct. 30, 2014; PCT Pat. Apl. Ser. No. PCT/US2015/026443, filed on Apr. 17, 2015, entitled “Systems and Methods for Barcoding Nucleic Acids”; U.S. Pat. Apl. Ser. No. 62/106,981, entitled “Systems, Methods, and Kits for Amplifying or Cloning Within Droplets,” by Weitz, et al.; U.S. Pat. Apl. Pub. No. 2010-0136544, entitled “Assay and Other Reactions Involving Droplets,” by Agresti, et al.; U.S. Pat. Apl. Ser. No. 61/981,108, entitled “Methods and Systems for Droplet Tagging and Amplification,” by Weitz, et al.; Int. Pat. Apl. Pub. No. PCT/US2014/037962, filed May 14, 2014, entitled “Rapid Production of Droplets,” by Weitz, et al.; and U.S. Provisional Patent Application Ser. No. 62/133,140, filed Mar. 13, 2015, entitled “Determination of Cells Using Amplification,” by Weitz, et al.

Furthermore, a patent application filed on Sep. 26, 2022, entitled “Systems and Methods for Screening of Large Gene Libraries,” U.S. Provisional Patent Application Ser. No. 63/410,140, by Weitz et al., is also incorporated herein by reference in its entirety. Also, U.S. Provisional Patent Application Ser. No. 63/410,116, filed Sep. 26, 2022, entitled “Methods and Systems for Full Gene Length Single Point Mutagenesis,” by Weitz, et al., is also incorporated herein by reference in its entirety.

The following examples are intended to illustrate certain embodiments of the present disclosure, but do not exemplify the full scope of the disclosure.

### Example 1

This example describes preparation of a DNA library using a method described herein, according to a non-limiting embodiment. A methylated circular DNA expressing esterase Lipase A (LipA, a 182 amino acid protein) was used as a common template nucleic acid. 18 pluralities of primers, each 10 codons in length, were ordered to cover nearly the full length of the common template nucleic acid.

Each plurality of primers included 10×19 primers. Each primer of a plurality was non-complementary to exactly one codon of the corresponding portion of the common template nucleic acid, and could thus be used to synthesize a nucleic acid differing from the common template nucleic acid at exactly one codon. Thus, each plurality of primers could be used to synthesize a corresponding plurality of up to 190 nucleic acids—the number of nucleic acids necessary to encode every LipA mutant including a single amino acid mutation in the portion of the LipA encoded by the portion of the common template nucleic acid corresponding to the primers.

The first plurality of primers corresponded to the first 10 codons of the common template nucleic acid; the second plurality corresponded to the second 10 codons of the common template nucleic acid, and so on. Pluralities of primers were pooled into 5 pools, to produce a five pluralities of nucleic acids that each included 3-4 point mutations of LipA. The first pool included primer pluralities 1, 7, 12, and 17; the second pool included primer pluralities 2, 8, 13, and 18; the third pool included primer pluralities 3, 9, and 14; the fourth pool included primer pluralities 4, 10, and 15; and the fifth primer pool included pluralities 5, 6, 11, and 16.

To introduce mutations, a reaction was performed using the QuikChange Lightning Multi Site-Directed Mutagenesis Kit (Agilent technologies), in a mixture of: 12.5 microliters of QuikChange Lightning Multi reaction buffer, 50 ng common template nucleic acid; 50 μmol of each plurality of primers; 0.5 microliters of QuikChange Lightning Multi enzyme blend; 0.5 microliters of dNTP mix; and 0.25 microliters of QuikSolution, with deionized water added to bring the reaction mixture to a total volume of 11.5 microliters.

Each pool was cycled to synthesize the plurality of nucleic acids, by a process of (i) holding the mixture at 95° C. for 2 minutes; (ii) iteratively holding the mixture at 95° C. for 20 seconds, holding at 55° C. for 30 seconds, and holding at 65° C. for 3 minutes, for a total of 30 iterations; and (iii) holding the mixture at 65° C. for 5 minutes. Then, the mixture was brought to 37° C., and 0.5 microliters of Dpn I restriction enzyme was added. The reaction was incubated at 37° C. for 2 hours, to digest the methylated common template nucleic acid to be digested.

Each pool was added to 50 microliters of ElectroTen-Blue Electroporation Competent Cells (Agilent Technology). Immediately after electroporation, microliters of sterile SOC medium was added.

After 1 hour of shaking at 37° C. and 250 rpm, 20 microliters of the resulting culture was plated on an LB-Trimethoprim agar plate as a control, and the remaining liquid culture was added to 3 mL of LB-Trimethoprim for overnight growth. The number of nucleic acids recovered from each pool was estimated to be around 1 million. Next, plasmids were isolated from the 5 cultures, mixed in an equimolar ratio, and diluted to a concentration of 1 ng/microliter. DNA shuffling was performed using Taq 2× Master Mix (New England Biolabs). The mixture included 2.5 microliters of pooled nucleic acids, 50 microliters of Taq 2× Master Mix, 1.5 microliters of 10 micromolar forward primer, 1.5 microliters of 10 micromolar reverse primer, and nuclease-free water in an amount sufficient to bring the total volume to 100 microliters. DNA shuffling was performed using 99 cycles of: (i) holding at 94° C. for 30 seconds and (ii) holding at 55° C. for 5 seconds.

After the thermocycling, the reaction mix was loaded to gel electrophoresis and DNA fragments with the size of a full gene were excised followed by purification using the Monarch® DNA Gel Extraction Kit (New England Biolabs). The purified DNA product was then used as megaprimers for overlap PCR to form a mutagenesis library construct on the desired plasmid backbone. The reaction mixture included 300 ng of Plasmid template with WT EstA gene insert, 25 microliters of Q5 High-Fidelity 2× Master Mix, 250 ng of purified megaprimer, and enough nuclease-free water to bring the total volume to 50 microliters. Overlap PCR was performed by a process of (i) holding the mixture at 98° C. for 2 minutes; (ii) iteratively holding the mixture at 98° C. for 10 seconds, holding at 61° C. for 20 seconds, and holding at 62° C. for 3 minutes, for a total of 30 iterations; and (iii) holding the mixture at 62° C. for 5 minutes.

Following the thermal cycling, the reaction was cooled down, and 1 microliter of Dpn I restriction enzyme was added. The reactions were incubated at 37° C. for 2 hours to ensure complete digestion of methylated parental DNA templates. The reaction was further purified with Monarch PCR & DNA Cleanup Kit (New England Biolabs) and eluted into 10 microliters of nuclease-free water, which was then transferred directly into 100 microliters of ElectroTen-Blue Electroporation Competent Cells (Agilent Technology). Immediately after electroporation, 900 μL of sterile SOC medium was added.

After 1 hour of shaking at 37° C. and 250 rpm, 20 microliters of the culture was plated on an LB-Trimethoprim agar plate as a control, and the remaining liquid culture was added to 3 mL of LB-Trimethoprim for overnight growth. The random library plasmid pool was isolated with Monarch Plasmid Miniprep Kit (NEB) and ready for downstream applications. The size of the library was estimated to include approximately 10⁸ nucleic acids.

This example demonstrates that very large libraries, including forbidden codon mutations not possible using conventional error-prone PCR, can be produced using the methods described herein.

### Example 2

This example demonstrates the use of the nucleic acid library of Example 1. In this example, the library was transformed using electrocompetent BW1125 cells. The ~10⁸ nucleic acids were grown to an optical density at 600 nm (OD600) of 0.5 to 0.7 and the cells were induced with arabinose at a final concentration of 0.004% for 16 hours at 28° C.

The cells were then washed with 1×PBS and suspended in a 1×PBS solution containing 50% Percoll for density matching. The final OD600 was calibrated to 0.08. The substrate, resorufin acetate, was diluted in 1×PBS to a final concentration of 7.5 nM.

A PDMS microfluidic device was made using standard soft lithographic methods for 25 μm droplet makers and 25 μm droplet sorters. Droplets were collected on ice to quench the activity and reinject into a droplet sorter at room temperature to pass through a laser. Each droplet passed through the same length of microfluidic tubing at room temperature, and hence the reaction of enzymes was incubated for the same time for all droplets. The fluorescence of each droplet was collected by a microscope objective and sent to a computer running a custom LabView program. Users set gates for droplet collection, which activated electrodes to create a gradient field that moved the droplets to the collection channel. A dynamic gate was set to sort the most active 0.1% of droplets, with the highest fluorescence signal. The cells in the droplets were collected and plated on an LB-Trimethoprim agar plate.

After overnight growth, single colonies were then picked and inoculated on a 96 well-plate. The sorted variants were then validated on a plate reader for their activity on resorufin acetate. A variant with a 6-fold increase in activity as compared to the wild-type enzyme was identified. FIG. 3 shows the normalized activity of a non-active variant of LipA, the wild-type (WT) LipA, and the mutated variant (N48S N51V) of LipA produced by the methods described herein. Moreover, one of the two mutations found in this variant had an N(AAC) to V(GTG) mutation, which is almost impossible with a round of error-prone PCR, meaning that it likely could not have been identified using a conventional nucleic acid library. Furthermore, the relative proximity of the two mutations in the protein (position 48 vs position 51) demonstrates the validity of the DNA shuffling approach, since these mutations could not have appeared simultaneously using the pooled primers as designed.

This result demonstrates that the method of Example 1 produced nucleic acid variants with useful properties, and that this approach is a viable and useful strategy for production of large nucleic acid libraries.

### Example 3

In this example, libraries were prepared using a process similar to that of Example 1, but using different numbers of primer pluralities in each pool of primers. One library was prepared using 18 separate pools, each including one primer plurality, followed by DNA shuffling and overlap PCR. Another library was produced using two pools, each including nine primer pluralities, followed by DNA shuffling and overlap PCR.

### Example 4

This example presents a similar method for preparation of large random mutagenesis libraries. The enzyme LipA (182 aa, though only 181 aa of the sequence was mutated) was used as a proof-of-concept common template nucleic acid, as in Example 1. Eighteen pluralities of primers each, covering 10 codons were ordered to cover the full LipA gene. As in the case of Example 1, each plurality of primers included 10×19 primers. Each primer of a plurality was non-complementary to exactly one codon of the corresponding portion of the common template nucleic acid, and could thus be used to synthesize a nucleic acid differing from the common template nucleic acid at exactly one codon. Thus, each plurality of primers, in isolation, could be used to synthesize a corresponding plurality of up to 190 nucleic acids—the number of nucleic acids necessary to encode every LipA mutant including a single amino acid mutation in the portion of the LipA encoded by the portion of the common template nucleic acid corresponding to the primers. Primers were ordered through Integrated DNA Technology (IDT).

The 18 pluralities of primers were used to create a variety of libraries of nucleic acid sequences by variations on the methods described in Example 1, above. During the preparation of each library of nucleic acid sequences, the steps of introducing mutations and cycling to synthesize the plurality of nucleic acids were performed as described in Example 1. As in Example 1, the initial reaction of the common template nucleic acid with pools of pluralities of primers to initially produce a number of nucleic acids that was estimated to be around 1 million.

To tailor the mutation rate and library type, various pluralities of primers were pooled into reactions. As a result, the plurality of nucleic acids synthesized in any given pool could include up to a number of mutations equal to the number of pluralities of nucleic acids in a pool. This allowed precise management of both the locations and rates of mutations across the target gene.

Three distinct libraries were created—labeled “random,” “3×6,” and “1×18”—to demonstrate different strategies for primer pooling. For the random library, pools were grouped as indicated in Table 1. For the 3×6 library, pools were grouped as indicated in Table 2. For the 1×18 library, pools were grouped as indicated in Table 3. The pluralities of primers are numbered based on the sequential position of each plurality of primers with respect to the codons of the common template nucleic acid. Thus, pool 1 contained the plurality of primers configured to bind to the first ten codons of the primary template nucleic acid and pool 2 contained the plurality of primers configured to bind to the second ten codons of the primary template nucleic acid.

TABLE 1
Pooling for random library preparation.

| Reaction | Pluralities |
|---|---|
| 1 | 1, 6, 11, and 16 |
| 2 | 2, 7, 12, and 17 |
| 3 | 3, 8, 13, and 18 |
| 4 | 4, 9, and 14 |
| 5 | 5, 10, and 15 |

TABLE 2
Pools for 3 × 6 library preparation.

| Reaction | Pluralities |
|---|---|
| 1 | 1, 4, 7, 10, 13, and 16 |
| 2 | 2, 5, 8, 11, 14, and 17 |
| 3 | 3, 6, 9, 12, 15, and 18 |

TABLE 3
Pools for 1 × 18 library preparation.

| Reaction | Pluralities |
|---|---|
| 1 | 1, 2, 3, 4, 5, 6, 7, 8, 9, 10, 11, |
| | 12, 13, 14, 15, 16, 17, and 18 |

After the site-specific mutagenesis reactions, an additional layer of variability was introduced by executing a round of DNA shuffling. DNA shuffling of the pools was performed as described in Example 1. A library prepared by error-prone PCR was used as a control. Samples were then linearized using a restriction enzyme (NruI-HF, NEB #R3192S) and sequenced using PacBio Sequel II by commercial providers (Icahn Institute for Genomics and Multiscale Biology). Between 20,000 to 115,000 reads were examined in each constructed library to assess the mutation rate.

Table 4 below provides a listing of the sequences of the primers used in each plurality of primers, as well as the sequence of the wild-type (WT) common template primer associated with the same portion of the common template nucleic acid. Other than WT sequences, each sequence in Table 4 associated with a plurality of primers connotes 19 primer sequences, each of which was present in the indicated plurality of primers. The wild-card codons (connoted by the “NNN” triplet of wild-card nucleotides) in each sequence were systematically mutated from the corresponding wild-type codon to each of the 19 codons in Table 5 that encoded a different amino acid than the corresponding wild-type codon. For example, a wild-type codon encoding arginine would be systematically replaced by each non-arginine-encoding codon of Table 5. It should be understood that the wild-card nucleotides “N” are interdependent to the extent that each wild-card codon “NNN” must be one of 19 possible codons in Table 5. Additionally, it should be understood that the Seq. ID. Nos. provided in each row of Table 4 are ordered such that the codons in the “NNN” position of each sequence are numbered in the order of appearance in Table 5. For example, Seq. ID. No. 2 has NNN=ATT, Seq. ID. No. 3 has NNN=ATG, etc., such that every codon in Table 5 except “GAA” appears because “GAA” encodes the same amino acid encoded by the WT-sequence. Table 6 below provides the amino acid sequence of the WT amino acid sequence and the sequence of the DNA encoding the WT amino acid sequence.

TABLE 4
Sequences of each plurality of primers. Sequence numbers in each non-WT
row refer to successive versions of the sequence listed in this table where, in each
sequence, “NNN” is replaced by the codons of Table 5 in the order presented in Table
5. The codon of Table 5 encoding the same amino acid as the codon of the WT
sequence is not numbered and was not used.

| | | Codon | |
|---|---|---|---|
| Plurality | Seq. ID. | Mutation | |
| No. | No(s). | Position | Sequence(s) |
| | 1 | WT | GAACACAACCCGGTTGTAATGGTTCATGGT |
| 1 | 2-20 | 1 | NNNCACAACCCGGTTGTAATGGTTCATGGT |
| 1 | 21-39 | 2 | GAANNNAACCCGGTTGTAATGGTTCATGGT |
| 1 | 40-58 | 3 | GAACACNNNCCGGTTGTAATGGTTCATGGT |
| 1 | 59-77 | 4 | GAACACAACNNNGTTGTAATGGTTCATGGT |
| 1 | 78-96 | 5 | GAACACAACCCGNNNGTAATGGTTCATGGT |
| 1 | 97-115 | 6 | GAACACAACCCGGTTNNNATGGTTCATGGT |
| 1 | 116-134 | 7 | GAACACAACCCGGTTGTANNNGTTCATGGT |
| 1 | 135-153 | 8 | GAACACAACCCGGTTGTAATGNNNCATGGT |
| 1 | 154-172 | 9 | GAACACAACCCGGTTGTAATGGTTNNNGGT |
| 1 | 173-191 | 10 | GAACACAACCCGGTTGTAATGGTTCATNNN |
| | 192 | WT | ATTGGCGGTGCCAGCTTCAACTTTGCGGGC |
| 2 | 193-211 | 11 | NNNGGCGGTGCCAGCTTCAACTTTGCGGGC |
| 2 | 212-230 | 12 | ATTNNNGGTGCCAGCTTCAACTTTGCGGGC |
| 2 | 231-249 | 13 | ATTGGCNNNGCCAGCTTCAACTTTGCGGGC |
| 2 | 250-268 | 14 | ATTGGCGGTNNNAGCTTCAACTTTGCGGGC |
| 2 | 269-287 | 15 | ATTGGCGGTGCCNNNTTCAACTTTGCGGGC |
| 2 | 288-306 | 16 | ATTGGCGGTGCCAGCNNNAACTTTGCGGGC |
| 2 | 307-325 | 17 | ATTGGCGGTGCCAGCTTCNNNTTTGCGGGC |
| 2 | 326-344 | 18 | ATTGGCGGTGCCAGCTTCAACNNNGCGGGC |
| 2 | 345-363 | 19 | ATTGGCGGTGCCAGCTTCAACTTTNNNGGC |
| 2 | 364-382 | 20 | ATTGGCGGTGCCAGCTTCAACTTTGCGNNN |
| | 383 | WT | ATCAAATCTTATCTGGTATCCCAGGGCTGG |
| 3 | 384-402 | 21 | NNNAAATCTTATCTGGTATCCCAGGGCTGG |
| 3 | 403-421 | 22 | ATCNNNTCTTATCTGGTATCCCAGGGCTGG |
| 3 | 422-440 | 23 | ATCAAANNNTATCTGGTATCCCAGGGCTGG |
| 3 | 441-459 | 24 | ATCAAATCTNNNCTGGTATCCCAGGGCTGG |
| 3 | 460-478 | 25 | ATCAAATCTTATNNNGTATCCCAGGGCTGG |
| 3 | 479-497 | 26 | ATCAAATCTTATCTGNNNTCCCAGGGCTGG |
| 3 | 498-516 | 27 | ATCAAATCTTATCTGGTANNNCAGGGCTGG |
| 3 | 517-535 | 28 | ATCAAATCTTATCTGGTATCCNNNGGCTGG |
| 3 | 536-554 | 29 | ATCAAATCTTATCTGGTATCCCAGNNNTGG |
| 3 | 555-573 | 30 | ATCAAATCTTATCTGGTATCCCAGGGCNNN |
| | 574 | WT | TCCCGTGATAAACTGTACGCAGTAGACTTC |
| 4 | 575-593 | 31 | NNNCGTGATAAACTGTACGCAGTAGACTTC |
| 4 | 594-612 | 32 | TCCNNNGATAAACTGTACGCAGTAGACTTC |
| 4 | 613-631 | 33 | TCCCGTNNNAAACTGTACGCAGTAGACTTC |
| 4 | 632-650 | 34 | TCCCGTGATNNNCTGTACGCAGTAGACTTC |
| 4 | 651-669 | 35 | TCCCGTGATAAANNNTACGCAGTAGACTTC |
| 4 | 670-688 | 36 | TCCCGTGATAAACTGNNNGCAGTAGACTTC |
| 4 | 689-707 | 37 | TCCCGTGATAAACTGTACNNNGTAGACTTC |
| 4 | 708-726 | 38 | TCCCGTGATAAACTGTACGCANNNGACTTC |
| 4 | 727-745 | 39 | TCCCGTGATAAACTGTACGCAGTANNNTTC |
| 4 | 746-764 | 40 | TCCCGTGATAAACTGTACGCAGTAGACNNN |
| | 765 | WT | TGGGACAAGACCGGCACCAACTACAATAAC |
| 5 | 766-784 | 41 | NNNGACAAGACCGGCACCAACTACAATAAC |
| 5 | 785-803 | 42 | TGGNNNAAGACCGGCACCAACTACAATAAC |
| 5 | 804-822 | 43 | TGGGACNNNACCGGCACCAACTACAATAAC |
| 5 | 823-841 | 44 | TGGGACAAGNNNGGCACCAACTACAATAAC |
| 5 | 842-860 | 45 | TGGGACAAGACCNNNACCAACTACAATAAC |
| 5 | 861-879 | 46 | TGGGACAAGACCGGCNNNAACTACAATAAC |
| 5 | 880-898 | 47 | TGGGACAAGACCGGCACCNNNTACAATAAC |
| 5 | 899-917 | 48 | TGGGACAAGACCGGCACCAACNNNAATAAC |
| 5 | 918-936 | 49 | TGGGACAAGACCGGCACCAACTACNNNAAC |
| 5 | 937-955 | 50 | TGGGACAAGACCGGCACCAACTACAATNNN |
| | 956 | WT | GGCCCGGTTCTGAGCCGTTTTGTCCAGAAA |
| 6 | 957-975 | 51 | NNNCCGGTTCTGAGCCGTTTTGTCCAGAAA |
| 6 | 976-994 | 52 | GGCNNNGTTCTGAGCCGTTTTGTCCAGAAA |
| 6 | 995-1013 | 53 | GGCCCGNNNCTGAGCCGTTTTGTCCAGAAA |
| 6 | 1014-1032 | 54 | GGCCCGGTTNNNAGCCGTTTTGTCCAGAAA |
| 6 | 1033-1051 | 55 | GGCCCGGTTCTGNNNCGTTTTGTCCAGAAA |
| 6 | 1052-1070 | 56 | GGCCCGGTTCTGAGCNNNTTTGTCCAGAAA |
| 6 | 1071-1089 | 57 | GGCCCGGTTCTGAGCCGTNNNGTCCAGAAA |
| 6 | 1090-1108 | 58 | GGCCCGGTTCTGAGCCGTTTTNNNCAGAAA |
| 6 | 1109-1127 | 59 | GGCCCGGTTCTGAGCCGTTTTGTCNNNAAA |
| 6 | 1128-1146 | 60 | GGCCCGGTTCTGAGCCGTTTTGTCCAGNNN |
| | 1147 | WT | GTGCTGGACGAAACTGGTGCTAAAAAGGTG |
| 7 | 1148-1166 | 61 | NNNCTGGACGAAACTGGTGCTAAAAAGGTG |
| 7 | 1167-1185 | 62 | GTGNNNGACGAAACTGGTGCTAAAAAGGTG |
| 7 | 1186-1204 | 63 | GTGCTGNNNGAAACTGGTGCTAAAAAGGTG |
| 7 | 1205-1223 | 64 | GTGCTGGACNNNACTGGTGCTAAAAAGGTG |
| 7 | 1224-1242 | 65 | GTGCTGGACGAANNNGGTGCTAAAAAGGTG |
| 7 | 1243-1261 | 66 | GTGCTGGACGAAACTNNNGCTAAAAAGGTG |
| 7 | 1262-1280 | 67 | GTGCTGGACGAAACTGGTNNNAAAAAGGTG |
| 7 | 1281-1299 | 68 | GTGCTGGACGAAACTGGTGCTNNNAAGGTG |
| 7 | 1300-1318 | 69 | GTGCTGGACGAAACTGGTGCTAAANNNGTG |
| 7 | 1319-1337 | 70 | GTGCTGGACGAAACTGGTGCTAAAAAGNNN |
| | 1338 | WT | GACATCGTTGCTCACAGCATGGGTGGCGCG |
| 8 | 1339-1357 | 71 | NNNATCGTTGCTCACAGCATGGGTGGCGCG |
| 8 | 1358-1376 | 72 | GACNNNGTTGCTCACAGCATGGGTGGCGCG |
| 8 | 1377-1395 | 73 | GACATCNNNGCTCACAGCATGGGTGGCGCG |
| 8 | 1396-1414 | 74 | GACATCGTTNNNCACAGCATGGGTGGCGCG |
| 8 | 1415-1433 | 75 | GACATCGTTGCTNNNAGCATGGGTGGCGCG |
| 8 | 1434-1452 | 76 | GACATCGTTGCTCACNNNATGGGTGGCGCG |
| 8 | 1453-1471 | 77 | GACATCGTTGCTCACAGCNNNGGTGGCGCG |
| 8 | 1472-1490 | 78 | GACATCGTTGCTCACAGCATGNNNGGCGCG |
| 8 | 1491-1509 | 79 | GACATCGTTGCTCACAGCATGGGTNNNGCG |
| 8 | 1510-1528 | 80 | GACATCGTTGCTCACAGCATGGGTGGCNNN |
| | 1529 | WT | AACACTCTGTATTACATCAAAAACCTGGAC |
| 9 | 1530-1548 | 81 | NNNACTCTGTATTACATCAAAAACCTGGAC |
| 9 | 1549-1567 | 82 | AACNNNCTGTATTACATCAAAAACCTGGAC |
| 9 | 1568-1586 | 83 | AACACTNNNTATTACATCAAAAACCTGGAC |
| 9 | 1587-1605 | 84 | AACACTCTGNNNTACATCAAAAACCTGGAC |
| 9 | 1606-1624 | 85 | AACACTCTGTATNNNATCAAAAACCTGGAC |
| 9 | 1625-1643 | 86 | AACACTCTGTATTACNNNAAAAACCTGGAC |
| 9 | 1644-1662 | 87 | AACACTCTGTATTACATCNNNAACCTGGAC |
| 9 | 1663-1681 | 88 | AACACTCTGTATTACATCAAANNNCTGGAC |
| 9 | 1682-1700 | 89 | AACACTCTGTATTACATCAAAAACNNNGAC |
| 9 | 1701-1719 | 90 | AACACTCTGTATTACATCAAAAACCTGNNN |
| | 1720 | WT | GGTGGCAACAAGGTAGCGAACGTTGTAACT |
| 10 | 1721-1739 | 91 | NNNGGCAACAAGGTAGCGAACGTTGTAACT |
| 10 | 1740-1758 | 92 | GGTNNNAACAAGGTAGCGAACGTTGTAACT |
| 10 | 1759-1777 | 93 | GGTGGCNNNAAGGTAGCGAACGTTGTAACT |
| 10 | 1778-1796 | 94 | GGTGGCAACNNNGTAGCGAACGTTGTAACT |
| 10 | 1797-1815 | 95 | GGTGGCAACAAGNNNGCGAACGTTGTAACT |
| 10 | 1816-1834 | 96 | GGTGGCAACAAGGTANNNAACGTTGTAACT |
| 10 | 1835-1853 | 97 | GGTGGCAACAAGGTAGCGNNNGTTGTAACT |
| 10 | 1854-1872 | 98 | GGTGGCAACAAGGTAGCGAACNNNGTAACT |
| 10 | 1873-1891 | 99 | GGTGGCAACAAGGTAGCGAACGTTNNNACT |
| 10 | 1892-1910 | 100 | GGTGGCAACAAGGTAGCGAACGTTGTANNN |
| | 1911 | WT | CTGGGTGGCGCTAACCGTCTGACCACTGGT |
| 11 | 1912-1930 | 101 | NNNGGTGGCGCTAACCGTCTGACCACTGGT |
| 11 | 1931-1949 | 102 | CTGNNNGGCGCTAACCGTCTGACCACTGGT |
| 11 | 1950-1968 | 103 | CTGGGTNNNGCTAACCGTCTGACCACTGGT |
| 11 | 1969-1987 | 104 | CTGGGTGGCNNNAACCGTCTGACCACTGGT |
| 11 | 1988-2006 | 105 | CTGGGTGGCGCTNNNCGTCTGACCACTGGT |
| 11 | 2007-2025 | 106 | CTGGGTGGCGCTAACNNNCTGACCACTGGT |
| 11 | 2026-2044 | 107 | CTGGGTGGCGCTAACCGTNNNACCACTGGT |
| 11 | 2045-2063 | 108 | CTGGGTGGCGCTAACCGTCTGNNNACTGGT |
| 11 | 2064-2082 | 109 | CTGGGTGGCGCTAACCGTCTGACCNNNGGT |
| 11 | 2083-2101 | 110 | CTGGGTGGCGCTAACCGTCTGACCACTNNN |
| | 2102 | WT | AAAGCGCTGCCGGGCACGGACCCGAACCAA |
| 12 | 2103-2121 | 111 | NNNGCGCTGCCGGGCACGGACCCGAACCAA |
| 12 | 2122-2140 | 112 | AAANNNCTGCCGGGCACGGACCCGAACCAA |
| 12 | 2141-2159 | 113 | AAAGCGNNNCCGGGCACGGACCCGAACCAA |
| 12 | 2160-2178 | 114 | AAAGCGCTGNNNGGCACGGACCCGAACCAA |
| 12 | 2179-2197 | 115 | AAAGCGCTGCCGNNNACGGACCCGAACCAA |
| 12 | 2198-2216 | 116 | AAAGCGCTGCCGGGCNNNGACCCGAACCAA |
| 12 | 2217-2235 | 117 | AAAGCGCTGCCGGGCACGNNNCCGAACCAA |
| 12 | 2236-2254 | 118 | AAAGCGCTGCCGGGCACGGACNNNAACCAA |
| 12 | 2255-2273 | 119 | AAAGCGCTGCCGGGCACGGACCCGNNNCAA |
| 12 | 2274-2292 | 120 | AAAGCGCTGCCGGGCACGGACCCGAACNNN |
| | 2293 | WT | AAAATCCTGTACACTTCCATCTACTCTTCC |
| 13 | 2294-2312 | 121 | NNNATCCTGTACACTTCCATCTACTCTTCC |
| 13 | 2313-2331 | 122 | AAANNNCTGTACACTTCCATCTACTCTTCC |
| 13 | 2332-2350 | 123 | AAAATCNNNTACACTTCCATCTACTCTTCC |
| 13 | 2351-2369 | 124 | AAAATCCTGNNNACTTCCATCTACTCTTCC |
| 13 | 2370-2388 | 125 | AAAATCCTGTACNNNTCCATCTACTCTTCC |
| 13 | 2389-2407 | 126 | AAAATCCTGTACACTNNNATCTACTCTTCC |
| 13 | 2408-2426 | 127 | AAAATCCTGTACACTTCCNNNTACTCTTCC |
| 13 | 2427-2445 | 128 | AAAATCCTGTACACTTCCATCNNNTCTTCC |
| 13 | 2446-2464 | 129 | AAAATCCTGTACACTTCCATCTACNNNTCC |
| 13 | 2465-2483 | 130 | AAAATCCTGTACACTTCCATCTACTCTNNN |
| | 2484 | WT | GCGGACATGATCGTTATGAATTACCTGTCC |
| 14 | 2485-2503 | 131 | NNNGACATGATCGTTATGAATTACCTGTCC |
| 14 | 2504-2522 | 132 | GCGNNNATGATCGTTATGAATTACCTGTCC |
| 14 | 2523-2541 | 133 | GCGGACNNNATCGTTATGAATTACCTGTCC |
| 14 | 2542-2560 | 134 | GCGGACATGNNNGTTATGAATTACCTGTCC |
| 14 | 2561-2579 | 135 | GCGGACATGATCNNNATGAATTACCTGTCC |
| 14 | 2580-2598 | 136 | GCGGACATGATCGTTNNNAATTACCTGTCC |
| 14 | 2599-2617 | 137 | GCGGACATGATCGTTATGNNNTACCTGTCC |
| 14 | 2618-2636 | 138 | GCGGACATGATCGTTATGAATNNNCTGTCC |
| 14 | 2637-2655 | 139 | GCGGACATGATCGTTATGAATTACNNNTCC |
| 14 | 2656-2674 | 140 | GCGGACATGATCGTTATGAATTACCTGNNN |
| | 2675 | WT | CGTCTGGATGGCGCTCGTAATGTACAGATC |
| 15 | 2676-2694 | 141 | NNNCTGGATGGCGCTCGTAATGTACAGATC |
| 15 | 2695-2713 | 142 | CGTNNNGATGGCGCTCGTAATGTACAGATC |
| 15 | 2714-2732 | 143 | CGTCTGNNNGGCGCTCGTAATGTACAGATC |
| 15 | 2733-2751 | 144 | CGTCTGGATNNNGCTCGTAATGTACAGATC |
| 15 | 2752-2770 | 145 | CGTCTGGATGGCNNNCGTAATGTACAGATC |
| 15 | 2771-2789 | 146 | CGTCTGGATGGCGCTNNNAATGTACAGATC |
| 15 | 2790-2808 | 147 | CGTCTGGATGGCGCTCGTNNNGTACAGATC |
| 15 | 2809-2827 | 148 | CGTCTGGATGGCGCTCGTAATNNNCAGATC |
| 15 | 2828-2846 | 149 | CGTCTGGATGGCGCTCGTAATGTANNNATC |
| 15 | 2847-2865 | 150 | CGTCTGGATGGCGCTCGTAATGTACAGNNN |
| | 2866 | WT | CACGGTGTCGGCCACATCGGTCTGCTGTAC |
| 16 | 2867-2885 | 151 | NNNGGTGTCGGCCACATCGGTCTGCTGTAC |
| 16 | 2886-2904 | 152 | CACNNNGTCGGCCACATCGGTCTGCTGTAC |
| 16 | 2905-2923 | 153 | CACGGTNNNGGCCACATCGGTCTGCTGTAC |
| 16 | 2924-2942 | 154 | CACGGTGTCNNNCACATCGGTCTGCTGTAC |
| 16 | 2943-2961 | 155 | CACGGTGTCGGCNNNATCGGTCTGCTGTAC |
| 16 | 2962-2980 | 156 | CACGGTGTCGGCCACNNNGGTCTGCTGTAC |
| 16 | 2981-2999 | 157 | CACGGTGTCGGCCACATCNNNCTGCTGTAC |
| 16 | 3000-3018 | 158 | CACGGTGTCGGCCACATCGGTNNNCTGTAC |
| 16 | 3019-3037 | 159 | CACGGTGTCGGCCACATCGGTCTGNNNTAC |
| 16 | 3038-3056 | 160 | CACGGTGTCGGCCACATCGGTCTGCTGNNN |
| | 3057 | WT | AGCTCCCAGGTGAACTCTCTGATTAAAGAG |
| 17 | 3058-3076 | 161 | NNNTCCCAGGTGAACTCTCTGATTAAAGAG |
| 17 | 3077-3095 | 162 | AGCNNNCAGGTGAACTCTCTGATTAAAGAG |
| 17 | 3096-3114 | 163 | AGCTCCNNNGTGAACTCTCTGATTAAAGAG |
| 17 | 3115-3133 | 164 | AGCTCCCAGNNNAACTCTCTGATTAAAGAG |
| 17 | 3134-3152 | 165 | AGCTCCCAGGTGNNNTCTCTGATTAAAGAG |
| 17 | 3153-3171 | 166 | AGCTCCCAGGTGAACNNNCTGATTAAAGAG |
| 17 | 3172-3190 | 167 | AGCTCCCAGGTGAACTCTNNNATTAAAGAG |
| 17 | 3191-3209 | 168 | AGCTCCCAGGTGAACTCTCTGNNNAAAGAG |
| 17 | 3210-3228 | 169 | AGCTCCCAGGTGAACTCTCTGATTNNNGAG |
| 17 | 3229-3247 | 170 | AGCTCCCAGGTGAACTCTCTGATTAAANNN |
| | 3248 | WT | GGTCTGAACGGCGGTGGCCAGAACACTAAC |
| 18 | 3249-3267 | 171 | NNNCTGAACGGCGGTGGCCAGAACACTAAC |
| 18 | 3268-3286 | 172 | GGTNNNAACGGCGGTGGCCAGAACACTAAC |
| 18 | 3287-3305 | 173 | GGTCTGNNNGGCGGTGGCCAGAACACTAAC |
| 18 | 3306-3324 | 174 | GGTCTGAACNNNGGTGGCCAGAACACTAAC |
| 18 | 3325-3343 | 175 | GGTCTGAACGGCNNNGGCCAGAACACTAAC |
| 18 | 3344-3362 | 176 | GGTCTGAACGGCGGTNNNCAGAACACTAAC |
| 18 | 3363-3381 | 177 | GGTCTGAACGGCGGTGGCNNNAACACTAAC |
| 18 | 3382-3400 | 178 | GGTCTGAACGGCGGTGGCCAGNNNACTAAC |
| 18 | 3401-3419 | 179 | GGTCTGAACGGCGGTGGCCAGAACNNNAAC |
| 18 | 3420-3438 | 180 | GGTCTGAACGGCGGTGGCCAGAACACTNNN |

TABLE 5
Codons substituted for wild-card “NNN” codons
of the sequences of Table 1

| Codon | Amino Acid Encoded |
|---|---|
| ATT | I |
| ATG | M |
| ACC | T |
| AAC | N |
| AAA | K |
| AGC | S |
| CGT | R |
| CTG | L |
| CCG | P |
| CAT | H |
| CAG | Q |
| GTG | V |
| GCG | A |
| GAT | D |
| GAA | E |
| GGC | G |
| TTT | F |
| TAT | Y |
| TGC | C |
| TGG | W |

TABLE 6
Wild-type lipase A amino acid sequence and encoding DNA sequence (both
sequences derive from B. subtilis).

| | Seq. Id. | |
|---|---|---|
| Name | No. | Sequence |
| Wild-Type LipA | 3439 | MEHNPVVMVHGIGGASFNFAGIKSYLVSQGWSRDKLYAV |
| (Amino Acid | | DFWDKTGTNYNNGPVLSRFVQKVLDETGAKKVDIVAHSM |
| Sequence) | | GGANTLYYIKNLDGGNKVANVVTLGGANRLTTGKALPGT |
| | | DPNQKILYTSIYSSADMIVMNYLSRLDGARNVQIHGVGHIG |
| | | LLYSSQVNSLIKEGLNGGGQNTN |
| Wild-Type LipA- | 3440 | ATGGAACACAACCCGGTTGTAATGGTTCATGGTATTGGC |
| encoding sequence | | GGTGCCAGCTTCAACTTTGCGGGCATCAAATCTTATCTG |
| (DNA Sequence) | | GTATCCCAGGGCTGGTCCCGTGATAAACTGTACGCAGTA |
| | | GACTTCTGGGACAAGACCGGCACCAACTACAATAACGG |
| | | CCCGGTTCTGAGCCGTTTTGTCCAGAAAGTGCTGGACGA |
| | | AACTGGTGCTAAAAAGGTGGACATCGTTGCTCACAGCAT |
| | | GGGTGGCGCGAACACTCTGTATTACATCAAAAACCTGGA |
| | | CGGTGGCAACAAGGTAGCGAACGTTGTAACTCTGGGTG |
| | | GCGCTAACCGTCTGACCACTGGTAAAGCGCTGCCGGGCA |
| | | CGGACCCGAACCAAAAAATCCTGTACACTTCCATCTACT |
| | | CTTCCGCGGACATGATCGTTATGAATTACCTGTCCCGTCT |
| | | GGATGGCGCTCGTAATGTACAGATCCACGGTGTCGGCCA |
| | | CATCGGTCTGCTGTACAGCTCCCAGGTGAACTCTCTGAT |
| | | TAAAGAGGGTCTGAACGGCGGTGGCCAGAACACTAAC |

Table 7 summarizes the results of the sequencing measurements, demonstrating the change in mutation rate observed within various pools. Table 7 compares the mutation rate observed after a single mutation step with the average mutations per gene of the random, 3×6, and 1×18 libraries and with the results of error-prone PCR (epPCR) generated using a commercial GeneMorph II kit from Agilent having a low mutation frequency of 0-4.5 mutations/kb. As shown in Table 7, the single mutation, random, 3×6 and 1×18 sequence libraries varied significantly in terms of the resulting number of mutations per gene, allowing a wide range of control over the mutation rate. At the extreme ends, the single mutation rate was almost identically 1, whereas the 1×18 library had a mutation rate of nearly 8 mutations per gene. The data quantitatively established that use of the methods provided herein offered a reliable and systematic way to control the mutation rate within the generated libraries.

TABLE 7
Mean mutations per gene, standard deviation in mutations
per gene, and sample size for various libraries

| | | | Mean Mutation | |
|---|---|---|---|---|
| | Mean | | Rate | Total |
| | Mutations | Standard | (mutations per | Measure- |
| Library | Per Gene | Deviation | kb per round) | ments |
| Single mutation | 0.99 | 0.67 | 1.8 | 42826 |
| Random mutation | 1.97 | 1.54 | 3.6 | 37441 |
| 3 × 6 | 5.09 | 2.15 | 9.4 | 45134 |
| 1 × 18 | 7.85 | 3.27 | 14.5 | 46789 |
| epPCR | 3.16 | 1.69 | 5.8 | 21376 |

Specifically, the number of mutations per gene corresponded with the number of pluralities of primer combined in a single reaction mix; as the number of pooled pluralities of nucleic acids increased, there was a corresponding and quantifiable increase in the mutation rate.

The frequency of early stop codons in each library was also examined. Early stop codons have the potential to prematurely terminate protein translation and render the resultant enzyme inactive. Early stop codons can occur when an amino acid—other than the terminal one—is randomly mutated into a stop codon. As shown in Table 8, libraries produced by the methods provided herein had a significantly reduced occurrence rate of early stop codons. In libraries generated using methods provided herein, the occurrence rate of stop codons ranged from 0.1% to 0.7%. In contrast, the epPCR library had an occurrence rate of early stop codons that was as high as 26.8%.

TABLE 8
Early stop codon occurrence rate for various libraries

| | Proportion of Nucleic Acids |
|---|---|
| Library | with Early Stop Codons |
| Single mutation | 0.1% |
| Random mutation | 0.7% |
| 3 × 6 | 0.7% |
| 1 × 18 | 0.7% |
| epPCR | 26.8% |

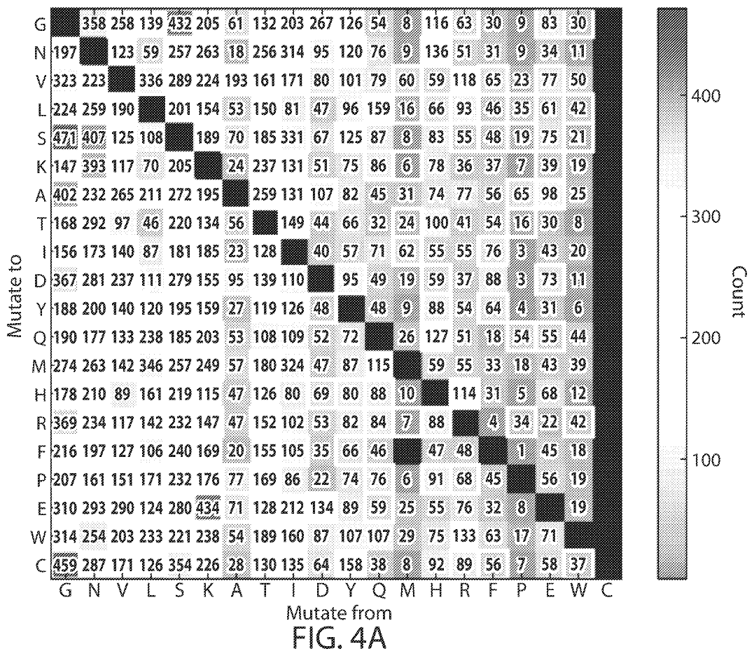

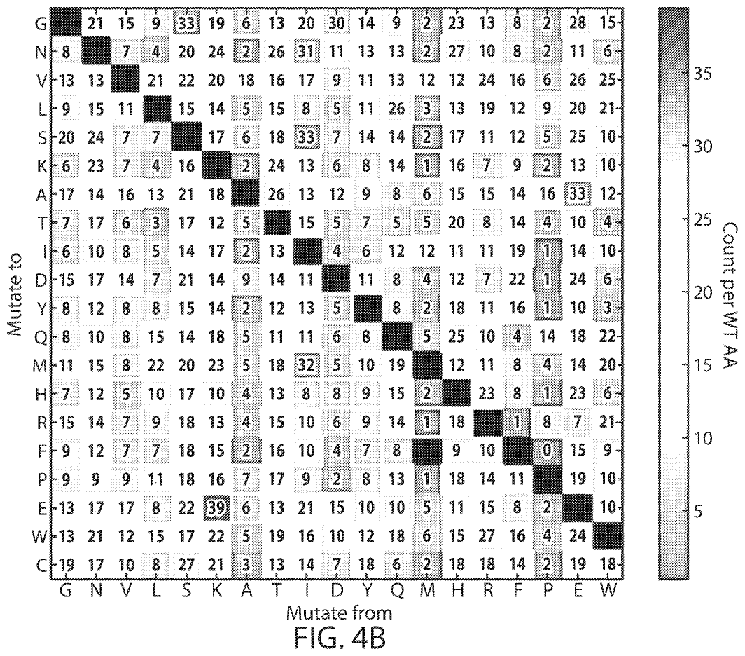

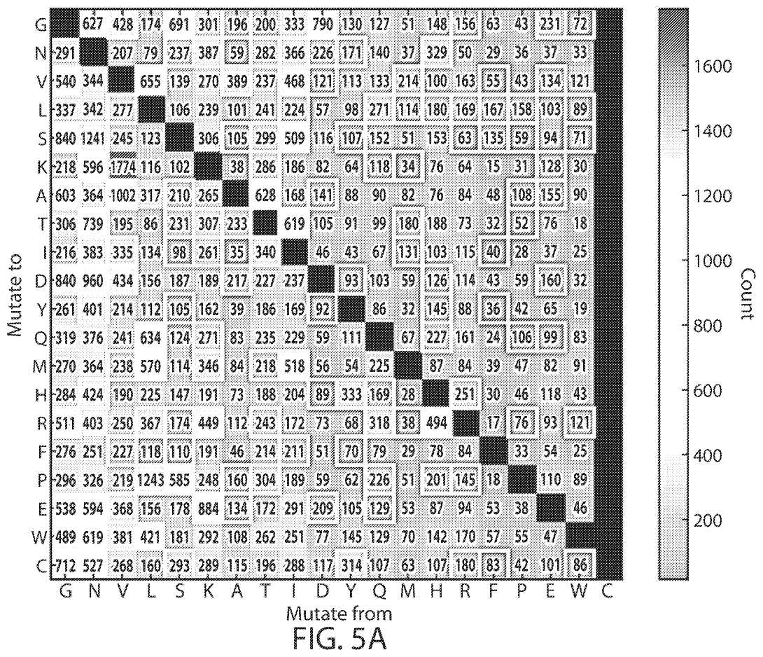

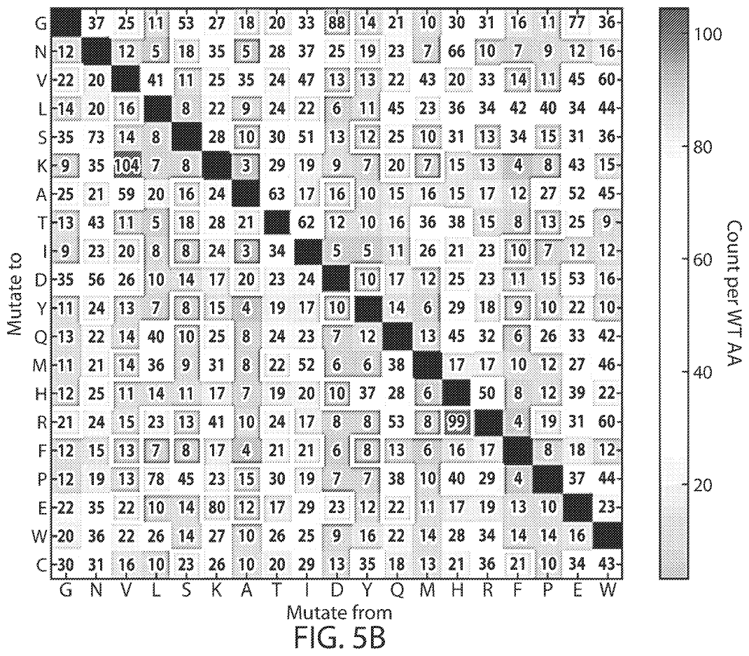

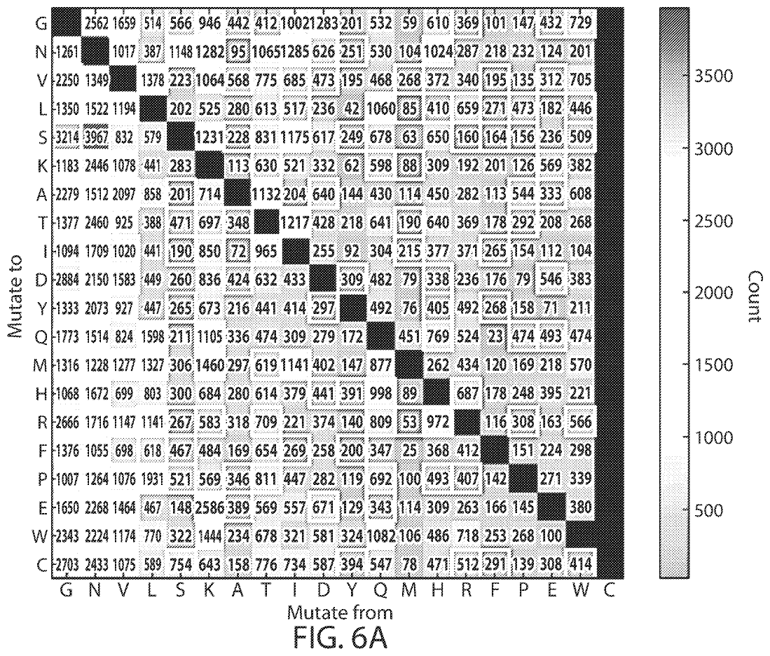

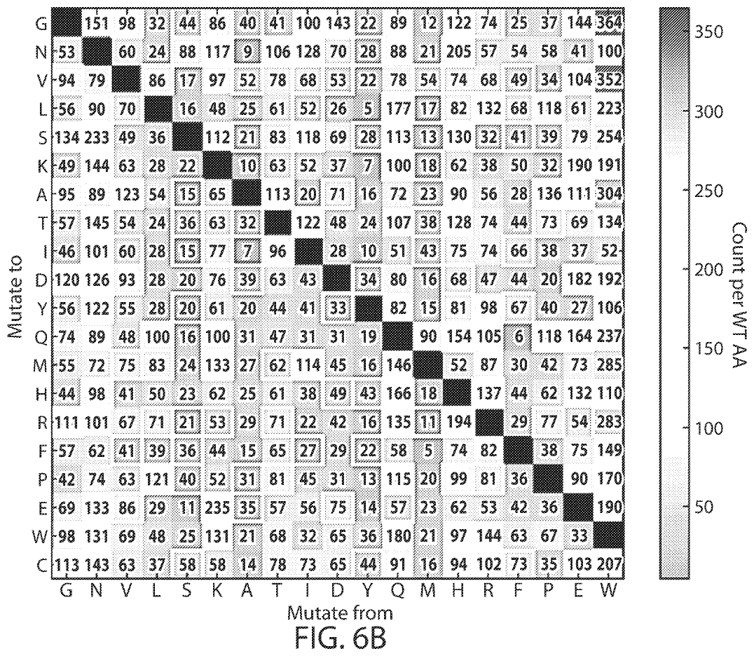

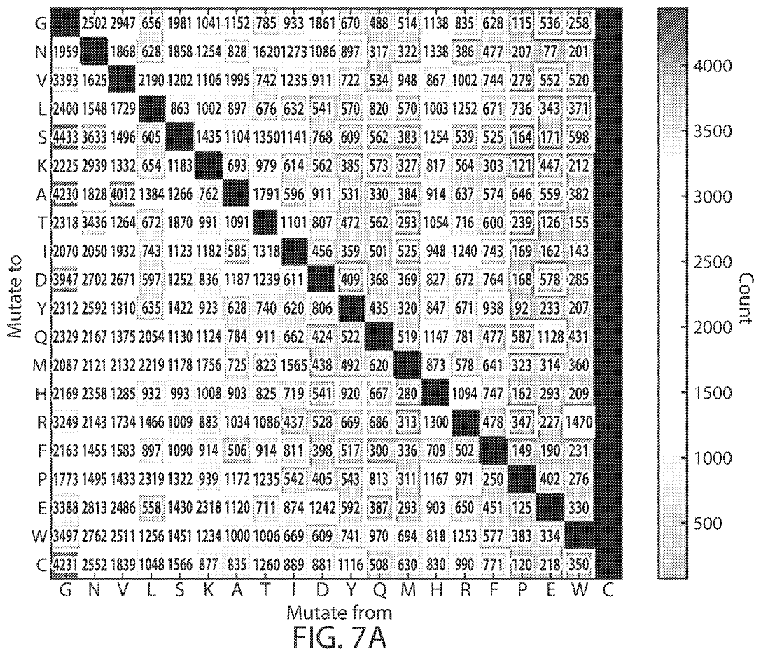

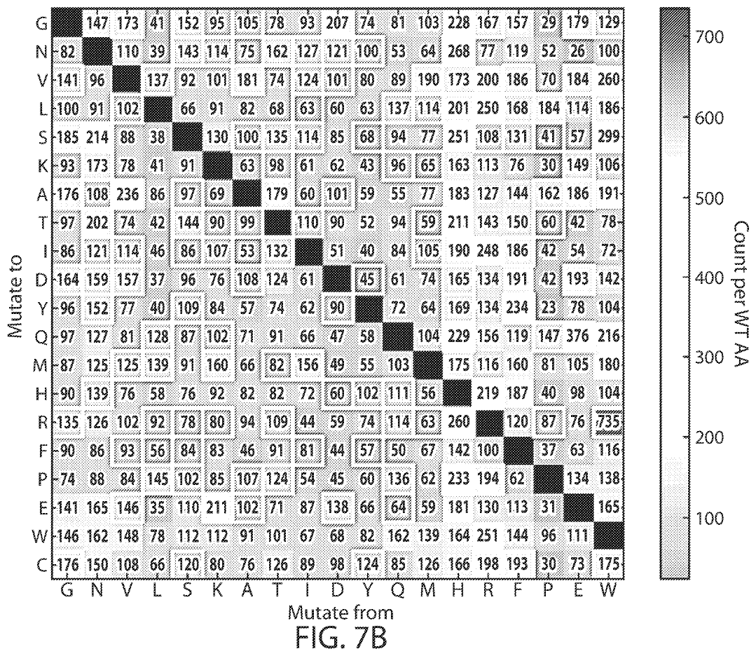

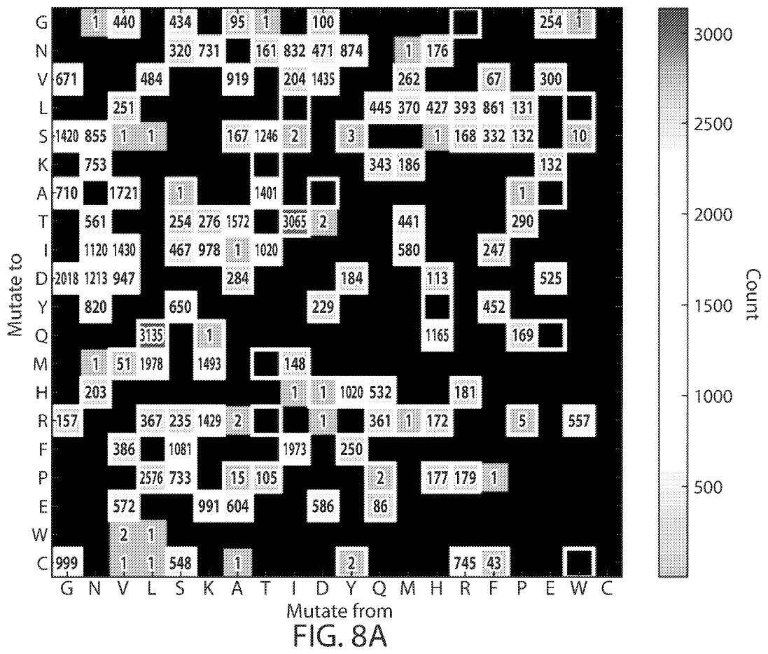

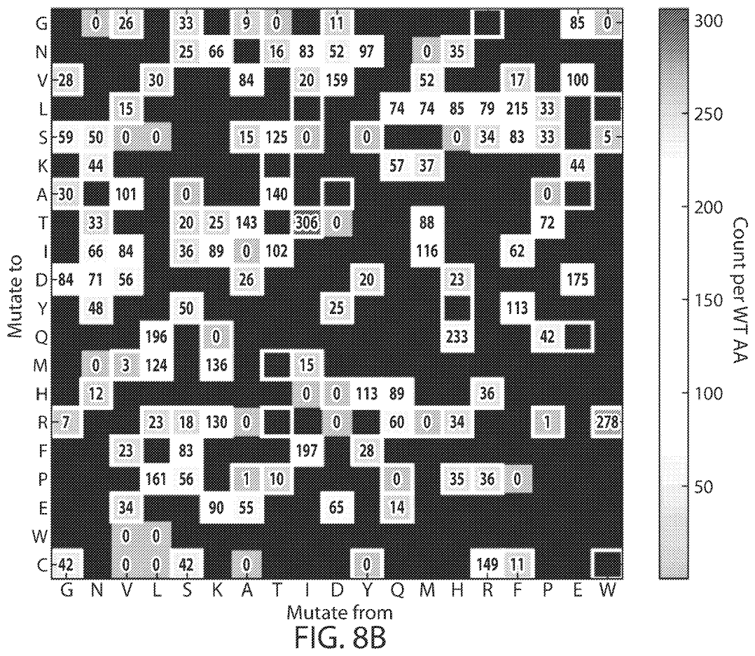

The mutations produced by the methods provided herein were also more diverse than those of the epPCR library. FIGS. 4A-8B provide heatmaps of the raw and normalized number of mutations of each amino acid observed in library. FIGS. 4A, 5A, 6A, 7A, and 8A show the raw number of mutations observed for each amino acid in LipA for the single mutation, random, 3×6, 1×18, and epPCR libraries, respectively. FIGS. 4B, 5B, 6B, 7B, and 8B show the raw number of mutations observed for each amino acid in LipA for the single mutation, normalized by the relative frequency of each amino acid in wild-type LipA. On the x-axis of each heat map, the amino acid corresponding to the original codon from which a mutation originates is shown, while the y-axis represents the codon into which it mutates. Dark boxes indicate that a particular codon mutation combination was not observed in the given library. Numerical values superimposed on light boxes indicate the numerical value observed for the raw or normalized mutation rate.

As shown, the single mutation, random, 3×6 and 1×18 libraries produced by methods provided herein resulted in the occurrence of almost every type of mutation—whereas the epPCR library produced less than half of all possible codon mutations. These results demonstrate the advantages of the methods provided herein, particularly over error prone PCR, for library preparation.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present disclosure.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, “wt %” is an abbreviation of weight percentage. As used herein, “at %” is an abbreviation of atomic percentage.

Some embodiments may be embodied as a method, of which various examples have been described. The acts performed as part of the methods may be ordered in any suitable way. Accordingly, embodiments may be constructed in which acts are performed in an order different than illustrated, which may include different (e.g., more or less) acts than those that are described, and/or that may involve performing some acts simultaneously, even though the acts are shown as being performed sequentially in the embodiments specifically described above.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The contents of the electronic sequence listing (H049870772WO00-SEQ-WRL.xml; Size: 3,072,001 bytes; and Date of Creation: Sep. 25, 2023) is herein incorporated by reference in its entirety.

What is claimed is:

1. A method of synthesizing a library of nucleic acid sequences, the method comprising:
synthesizing a first plurality of nucleic acids using a common template nucleic acid with a first plurality of primers; and
synthesizing a second plurality of nucleic acids using one or more nucleic acids of the first plurality of nucleic acids;
wherein the primers of the first plurality of primers define a first common template primer such that greater than or equal to 90% of the primers of the first plurality of primers differs from the first common template primer at exactly one codon; and
wherein at least 5 consecutive nucleotides of the common template nucleic acid are complimentary to at least 5 consecutive nucleotides of the first common template primer.

2. The method of claim 1, wherein synthesizing the second plurality of nucleic acids comprises nucleic acid shuffling and/or overlap amplification.

3. The method of claim 1, wherein the common template primer is a first common template primer,
wherein synthesizing the second plurality of nucleic acids comprises using a second plurality of primers to synthesize the second plurality of nucleic acids, wherein at least 5 consecutive nucleotides of the common template nucleic acid are complimentary to at least 5 consecutive nucleotides of a second common template primer, which are not complementary to the same portion of the common template nucleic acid as the first common template primer, and
wherein greater than or equal to 90% of the primers of the second plurality of primers differ from a second common template primer.

4. A method of synthesizing a library of nucleic acid sequences, the method comprising:
synthesizing a plurality of nucleic acids using a common template nucleic acid with a first plurality of primers and a second plurality of primers;
wherein the primers of the first plurality of primers define a first common template primer such that greater than or equal to 90% of the primers of the first plurality of primers differs from the first common template primer at exactly one codon;
wherein the primers of the second plurality of primers define a second common template primer such that greater than or equal to 90% of the primers of the second plurality of primers differs from the second common template primer at exactly one codon; and
wherein at least 5 consecutive nucleotides of the common template nucleic acid are complimentary to at least 5 consecutive nucleotides of the first common template primer, and at least at least 5 consecutive nucleotides of the common template nucleic acid are complimentary to at least 5 consecutive nucleotides of the second common template primer, which are not complementary to the same portion of the common template nucleic acid as the first common template primer.

5. A method of synthesizing a library of nucleic acid sequences, the method comprising:
synthesizing a first plurality of nucleic acids using a common template nucleic acid with a first plurality of primers;
synthesizing a second plurality of nucleic acids using the common template nucleic acid with a second plurality of primers; and
synthesizing a third plurality of nucleic acids using the first plurality of nucleic acids and the second plurality of nucleic acids,
wherein the primers of the first plurality of primers defines a first common template primer such that greater than or equal to 90% of the primers of the first plurality of primers differs from the first common template primer at exactly one codon; and
wherein the primers of the second plurality of primers defines a second common template primer such that greater than or equal to 90% of the primers of the second plurality of primers differs from the second common template primer at exactly one codon.

6. The method of claim 5, wherein at least 5 consecutive nucleotides of the common template nucleic acid are complimentary to at least 5 consecutive nucleotides of the first common template primer, and at least at least 5 consecutive nucleotides of the common template nucleic acid are complimentary to at least 5 consecutive nucleotides of the second common template primer, which are not complementary to the same portion of the common template nucleic acid as the first common template primer.

7. The method of claim 5, wherein the synthesizing the third plurality of nucleic acids comprises mixing and amplifying the first plurality of nucleic acids and the second plurality of nucleic acids.

8. The method of claim 5, wherein synthesizing the third plurality of nucleic acids comprises nucleic acid shuffling and/or overlap amplification.

9. The method of any preceding claim, wherein the method further comprises purifying the first plurality of nucleic acids.

10. The method of any preceding claim, wherein the method further comprises using less than or equal to 5 nucleic acids of the first plurality of nucleic acids to synthesize the second plurality of nucleic acids.

11. The method of any preceding claim, wherein the method further comprises identifying a subset of the first plurality of nucleic acids and using the subset of the first plurality of nucleic acids to synthesize the second plurality of nucleic acids.

12. The method of any preceding claim, wherein identifying the subset of the first plurality of nucleic acids comprises expressing the nucleic acids in plasmids.

13. The method of any preceding claim, wherein the common template nucleic acid, the first plurality of nucleic acids, and/or the second plurality of nucleic acids are circular nucleic acids.

14. The method of any preceding claim, wherein the synthesis of the first plurality of nucleic acids and/or synthesis of the second plurality of nucleic acids comprises nucleic acid amplification.

15. The method of any preceding claim, wherein the nucleic acid amplification is performed using PCR.

16. The method of any one of claims 1-3 and 5-15, wherein the first plurality of nucleic acids is contained within a droplet.

17. The method of any one of claims 1-3 and 5-15, wherein the first plurality of nucleic acids is contained within a plurality of separate droplets.

18. The method of claim 4, wherein the plurality of nucleic acids is contained within a droplet.

19. The method of claim 4, wherein the plurality of nucleic acids is contained within a plurality of separate droplets.

20. The method of any preceding claim, wherein the first plurality of primers comprises at least 15 unique primers.

21. The method of any preceding claim, wherein the first plurality of primers comprises at least 19 unique primers.

22. A library of nucleic acid sequences synthesized by the method of any one of the preceding claims, wherein the library of nucleic acid sequences comprises greater than or equal to 10⁶ nucleic acids.

23. A library of nucleic acid sequences synthesized by the method of any one of the preceding claims, wherein the library of nucleic acid sequences comprises greater than or equal to 10¹² nucleic acids.

24. The method of any one of the preceding claims, wherein at least one synthesis step has a maximum mutation rate of greater than or equal to 18 mutations per kb.

25. A solution, comprising:
a plurality of nucleic acids, wherein greater than or equal to 50% of the nucleic acids each differs from a common template nucleic acid at at least one codon; and
a plurality of primers, wherein greater than or equal to 50% of the primers of the plurality of primers differ from a common template primer at exactly one codon,
wherein at least 5 consecutive nucleotides of the common template nucleic acid are complimentary to at least 5 consecutive nucleotides of the common template primer;
wherein at least 90% of the plurality of primers have a structure such that the exactly one codon of the primer is not complementary to a sequentially corresponding codon of the common template nucleic acid.

26. The solution of any preceding claim, wherein the solution is aqueous.

27. The solution of any preceding claim, wherein the common template nucleic acid, and the plurality of nucleic acids are circular nucleic acids.

28. The solution of any preceding claim, wherein the plurality of primers comprises at least 15 unique primers.

29. The solution of any preceding claim, wherein the plurality of primers comprises at least 19 unique primers.

30. A method of synthesizing a library of nucleic acid sequences, the method comprising:
synthesizing a first plurality of nucleic acids using a common template nucleic acid with a first plurality of primers; and
synthesizing a second plurality of nucleic acids using one or more nucleic acids of the first plurality of nucleic acids;
wherein the primers of the first plurality of primers define a first common template primer such that greater than or equal to 90% of the primers of the first plurality of primers differs from the first common template primer at greater than or equal to 1 and less than or equal to 4 codons; and
wherein at least 5 consecutive nucleotides of the common template nucleic acid are complimentary to at least 5 consecutive nucleotides of the first common template primer.

31. The method of claim 30, wherein synthesizing the second plurality of nucleic acids comprises nucleic acid shuffling and/or overlap amplification.

32. The method of claim 30, wherein the common template primer is a first common template primer,
wherein synthesizing the second plurality of nucleic acids comprises using a second plurality of primers to synthesize the second plurality of nucleic acids, wherein at least 5 consecutive nucleotides of the common template nucleic acid are complimentary to at least 5 consecutive nucleotides of a second common template primer, which are not complementary to the same portion of the common template nucleic acid as the first common template primer, and
wherein greater than or equal to 90% of the primers of the second plurality of primers differ from a second common template primer.

33. The method of any one of claims 30-32, wherein greater than or equal to 90% of the primers of the first plurality of primers differs from the first common template primer at greater than or equal to 1 and less than or equal to 3 codons.

34. The method of any one of claims 30-33, wherein greater than or equal to 90% of the primers of the first plurality of primers differs from the first common template primer at greater than or equal to 1 and less than or equal to 2 codons.

35. The method of any one of claims 30-34, wherein greater than or equal to 90% of the primers of the first plurality of primers differs from the first common template primer at exactly 1 codon.

36. The method of any one of claims 30-34, wherein greater than or equal to 90% of the primers of the first plurality of primers differs from the first common template primer at exactly 2 codons.

37. A method of synthesizing a library of nucleic acid sequences, the method comprising:
synthesizing a plurality of nucleic acids using a common template nucleic acid with a first plurality of primers and a second plurality of primers;
wherein the primers of the first plurality of primers define a first common template primer such that greater than or equal to 90% of the primers of the first plurality of primers differs from the first common template primer at greater than or equal to 1 and less than or equal to 4 codons;
wherein the primers of the second plurality of primers define a second common template primer such that greater than or equal to 90% of the primers of the second plurality of primers differs from the second common template primer at at greater than or equal to 1 and less than or equal to 4 codons; and
wherein at least 5 consecutive nucleotides of the common template nucleic acid are complimentary to at least 5 consecutive nucleotides of the first common template primer, and at least at least 5 consecutive nucleotides of the common template nucleic acid are complimentary to at least 5 consecutive nucleotides of the second common template primer, which are not complementary to the same portion of the common template nucleic acid as the first common template primer.

38. The method of claim 37, wherein greater than or equal to 90% of the primers of the first plurality of primers differs from the first common template primer at greater than or equal to 1 and less than or equal to 3 codons.

39. The method of any one of claims 37-38, wherein greater than or equal to 90% of the primers of the first plurality of primers differs from the first common template primer at greater than or equal to 1 and less than or equal to 2 codons.

40. The method of any one of claims 37-39, wherein greater than or equal to 90% of the primers of the first plurality of primers differs from the first common template primer at exactly 1 codon.

41. The method of any one of claims 37-39, wherein greater than or equal to 90% of the primers of the first plurality of primers differs from the first common template primer at exactly 2 codons.

42. The method of any one of claims 37-41, wherein greater than or equal to 0% of the primers of the second plurality of primers differs from the second common template primer at greater than or equal to 1 and less than or equal to 3 codons.

43. The method of any one of claims 37-42, wherein greater than or equal to 90% of the primers of the second plurality of primers differs from the second common template primer at greater than or equal to 1 and less than or equal to 2 codons.

44. The method of any one of claims 37-43, wherein greater than or equal to 90% of the primers of the second plurality of primers differs from the second common template primer at exactly 1 codon.

45. The method of any one of claims 37-43, wherein greater than or equal to 90% of the primers of the second plurality of primers differs from the second common template primer at exactly 2 codons.

46. A method of synthesizing a library of nucleic acid sequences, the method comprising:
synthesizing a first plurality of nucleic acids using a common template nucleic acid with a first plurality of primers;
synthesizing a second plurality of nucleic acids using the common template nucleic acid with a second plurality of primers; and
synthesizing a third plurality of nucleic acids using the first plurality of nucleic acids and the second plurality of nucleic acids,
wherein the primers of the first plurality of primers defines a first common template primer such that greater than or equal to 90% of the primers of the first plurality of primers differs from the first common template primer at at greater than or equal to 1 and less than or equal to 4 codons; and
wherein the primers of the second plurality of primers defines a second common template primer such that greater than or equal to 90% of the primers of the second plurality of primers differs from the second common template primer at greater than or equal to 1 and less than or equal to 4 codons.

47. The method of claim 46, wherein greater than or equal to 90% of the primers of the first plurality of primers differs from the first common template primer at greater than or equal to 1 and less than or equal to 3 codons.

48. The method of any one of claims 46-47, wherein greater than or equal to 90% of the primers of the first plurality of primers differs from the first common template primer at greater than or equal to 1 and less than or equal to 2 codons.

49. The method of any one of claims 46-48, wherein greater than or equal to 90% of the primers of the first plurality of primers differs from the first common template primer at exactly 1 codon.

50. The method of any one of claims 46-48, wherein greater than or equal to 90% of the primers of the first plurality of primers differs from the first common template primer at exactly 2 codons.

51. The method of any one of claims 46-50, wherein greater than or equal to 0% of the primers of the second plurality of primers differs from the second common template primer at greater than or equal to 1 and less than or equal to 3 codons.

52. The method of any one of claims 46-51, wherein greater than or equal to 90% of the primers of the second plurality of primers differs from the second common template primer at greater than or equal to 1 and less than or equal to 2 codons.

53. The method of any one of claims 46-52, wherein greater than or equal to 90% of the primers of the second plurality of primers differs from the second common template primer at exactly 1 codon.

54. The method of any one of claims 46-52, wherein greater than or equal to 90% of the primers of the second plurality of primers differs from the second common template primer at exactly 2 codons.

55. The method of any one of claims 46-54, wherein at least 5 consecutive nucleotides of the common template nucleic acid are complimentary to at least 5 consecutive nucleotides of the first common template primer, and at least at least 5 consecutive nucleotides of the common template nucleic acid are complimentary to at least 5 consecutive nucleotides of the second common template primer, which are not complementary to the same portion of the common template nucleic acid as the first common template primer.

56. The method of any one of claims 46-54, wherein the synthesizing the third plurality of nucleic acids comprises mixing and amplifying the first plurality of nucleic acids and the second plurality of nucleic acids.

57. The method of any one of claims 46-54, wherein synthesizing the third plurality of nucleic acids comprises nucleic acid shuffling and/or overlap amplification.

58. The method of any one of claims 30-57, wherein the method further comprises purifying the first plurality of nucleic acids.

59. The method of any one of claims 30-58, wherein the method further comprises using less than or equal to 5 nucleic acids of the first plurality of nucleic acids to synthesize the second plurality of nucleic acids.

60. The method of any one of claims 30-59, wherein the method further comprises identifying a subset of the first plurality of nucleic acids and using the subset of the first plurality of nucleic acids to synthesize the second plurality of nucleic acids.

61. The method of any one of claims 30-60, wherein identifying the subset of the first plurality of nucleic acids comprises expressing the nucleic acids in plasmids.

62. The method of any one of claims 30-61, wherein the common template nucleic acid, the first plurality of nucleic acids, and/or the second plurality of nucleic acids are circular nucleic acids.

63. The method of any one of claims 30-62, wherein the synthesis of the first plurality of nucleic acids and/or synthesis of the second plurality of nucleic acids comprises nucleic acid amplification.

64. The method of any one of claims 30-63, wherein the nucleic acid amplification is performed using PCR.

65. The method of any one of claims 30-32 and 46-64, wherein the first plurality of nucleic acids is contained within a droplet.

66. The method of any one of claims 30-32 and 46-65, wherein the first plurality of nucleic acids is contained within a plurality of separate droplets.

67. The method of any one of claims 37-45, wherein the plurality of nucleic acids is contained within a droplet.

68. The method of any one of claims 37-45, wherein the plurality of nucleic acids is contained within a plurality of separate droplets.

69. The method of any one of claims 30-68, wherein the first plurality of primers comprises at least 15 unique primers.

70. The method of any one of claims 30-69, wherein the first plurality of primers comprises at least 19 unique primers.

71. A library of nucleic acid sequences synthesized by the method of any one of claims 30-70, wherein the library of nucleic acid sequences comprises greater than or equal to 10⁶ nucleic acids.

72. A library of nucleic acid sequences synthesized by the method of any one of claims 30-70, wherein the library of nucleic acid sequences comprises greater than or equal to 10¹² nucleic acids.

73. The method of any one of claims 30-70, wherein at least one synthesis step has a maximum mutation rate of greater than or equal to 18 mutations per kb.

74. A solution, comprising:
a plurality of nucleic acids, wherein greater than or equal to 50% of the nucleic acids each differs from a common template nucleic acid at at least one codon; and
a plurality of primers, wherein greater than or equal to 50% of the primers of the plurality of primers differ from a common template primer at greater than or equal to 1 and less than or equal to 4 codons,
wherein at least 5 consecutive nucleotides of the common template nucleic acid are complimentary to at least 5 consecutive nucleotides of the common template primer;
wherein at least 90% of the plurality of primers have a structure such that the at greater than or equal to 1 and less than or equal to 4 codons of the primer are not complementary to a sequentially corresponding codons of the common template nucleic acid.

75. The solution of claim 74, wherein the solution is aqueous.

76. The solution of any one of claims 74-75, wherein the common template nucleic acid, and the plurality of nucleic acids are circular nucleic acids.

77. The solution of any one of claims 74-76, wherein the plurality of primers comprises at least 15 unique primers.

78. The solution of any one of claims 74-77, wherein the plurality of primers comprises at least 19 unique primers.

79. The solution of any one of claims 74-78, wherein greater than or equal to 90% of the primers of the plurality of primers differs from the common template primer at greater than or equal to 1 and less than or equal to 3 codons.

80. The solution of any one of claims 74-79, wherein greater than or equal to 90% of the primers of the plurality of primers differs from the common template primer at greater than or equal to 1 and less than or equal to 2 codons.

81. The solution of any one of claims 74-80, wherein greater than or equal to 90% of the primers of the plurality of primers differs from the common template primer at exactly 1 codon.

82. The solution of any one of claims 74-81, wherein greater than or equal to 90% of the primers of the plurality of primers differs from the common template primer at exactly 2 codons.
